SEQUENCE LISTING (149 sequences)

SEQ ID NO: 1 - protein, 851 aa, Artificial Sequence
  TALE domain of T1L20.5
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALESIVAQLSRPDPALAAL

SEQ ID NO: 2 - protein, 197 aa, Artificial Sequence
  FokI nuclease domain of T1L20.5
LVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 3 - protein, 1074 aa, Artificial Sequence
  T1L20.5 TEN
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNGGGKQALESIVAQLSRPDPALAALLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 4 - protein, 715 aa, Artificial Sequence
  TALE domian of T2L16.5
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALESIVAQLSRPDPALAAL

SEQ ID NO: 5 - protein, 197 aa, Artificial Sequence; identical to SEQ ID NO: 2
  FokI nuclease domian of T2L16.5

SEQ ID NO: 6 - protein, 938 aa, Artificial Sequence
  T2L16.5 TEN
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNIGGKQALETVQRLLPVLCQAHGLTPEQVVAIASNGGGKQALETVQRLLPVLCQAHGLTPEQVVAIASHDGGKQALESIVAQLSRPDPALAALLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 7 - protein, 783 aa, Artificial Sequence
  TALE domain of T2R18.5
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALESIVAQLSRPDPALAAL

SEQ ID NO: 8 - protein, 197 aa, Artificial Sequence; identical to SEQ ID NO: 2
  FokI nuclease domain of T2R18.5

SEQ ID NO: 9 - protein, 1006 aa, Artificial Sequence
  T2R18.5 TEN
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPEQVVAIASNNGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNNGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALESIVAQLSRPDPALAALLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 10 - protein, 8 aa, Artificial Sequence
  NLS(nuclear localization signal)
PPKKKRKV

SEQ ID NO: 11 - DNA, 31 bp, Artificial Sequence
  AB-F primer
ttcgaattcaaatggatcccattcgttcgcg

SEQ ID NO: 12 - DNA, 31 bp, Artificial Sequence
  AB-R primer
ttgctcgagtcactgaggcaatagctccatc

SEQ ID NO: 13 - DNA, 23 bp, Artificial Sequence
  AB-N153F primer
ttcgaattcaagatctacgcacg

SEQ ID NO: 14 - DNA, 23 bp, Artificial Sequence
  AB-N254F primer
ttcgaattcaattggacacaggc

SEQ ID NO: 15 - DNA, 23 bp, Artificial Sequence
  AB-N285F primer
ttcgaattcaacccctgaacctg

SEQ ID NO: 16 - DNA, 23 bp, Artificial Sequence
  AB-C99R primer
ttactcgagtcagctgcttgccc

SEQ ID NO: 17 - DNA, 24 bp, Artificial Sequence
  AB-C263R primer
ttgctcgagcaacgcggccaacgc

SEQ ID NO: 18 - DNA, 39 bp, Artificial Sequence
  UPA20F primer
aattcatctttatataaacctgaccctttgtgacgagct

SEQ ID NO: 19 - DNA, 31 bp, Artificial Sequence
  UPA20R primer
cgtcacaaagggtcaggtttatataaagatg

SEQ ID NO: 20 - DNA, 108 bp, Artificial Sequence
  HD module
tctagagaccgtgcagcgcctgctgcccgtgctgtgccaggcccacggcctgacccccgagcaggtggtggccatcgccagccacgacggcggcaagcaggcgctagc

SEQ ID NO: 21 - DNA, 108 bp, Artificial Sequence
  NG module
tctagagaccgtgcagcgcctgctgcccgtgctgtgccaggcccacggcctgacccccgagcaggtggtggccatcgccagcaatggcggcggcaagcaggcgctagc

SEQ ID NO: 22 - DNA, 108 bp, Artificial Sequence
  NI module
tctagagaccgtgcagcgcctgctgcccgtgctgtgccaggcccacggcctgacccccgagcaggtggtggccatcgccagcaatattggcggcaagcaggcgctagc

SEQ ID NO: 23 - DNA, 108 bp, Artificial Sequence
  NN module
tctagagaccgtgcagcgcctgctgcccgtgctgtgccaggcccacggcctgacccccgagcaggtggtggccatcgccagcaataacggcggcaagcaggcgctagc

SEQ ID NO: 24 - protein, 34 aa, Artificial Sequence
  HD module
LTPEQVVAIASHDGGKQALETVQRLLPVLCQAHG

SEQ ID NO: 25 - protein, 34 aa, Artificial Sequence
  NG module
LTPEQVVAIASNGGGKQALETVQRLLPVLCQAHG

SEQ ID NO: 26 - protein, 34 aa, Artificial Sequence
  NI module
LTPEQVVAIASNIGGKQALETVQRLLPVLCQAHG

SEQ ID NO: 27 - protein, 34 aa, Artificial Sequence
  NN module
LTPEQVVAIASNNGGKQALETVQRLLPVLCQAHG

SEQ ID NO: 28 - protein, 135 aa, Artificial Sequence
  part of TALE domain
DLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 29 - protein, 15 aa, Artificial Sequence
  HQ linker
PALAALTNDHQLVKS

SEQ ID NO: 30 - protein, 14 aa, Artificial Sequence
  DQ linker
PALAALTNDQLVKS

SEQ ID NO: 31 - protein, 13 aa, Artificial Sequence
  NQ linker
PALAALTNQLVKS

SEQ ID NO: 32 - protein, 12 aa, Artificial Sequence
  TQ linker
PALAALTQLVKS

SEQ ID NO: 33 - protein, 11 aa, Artificial Sequence
  LQ linker
PALAALQLVKS

SEQ ID NO: 34 - protein, 10 aa, Artificial Sequence
  LL linker
PALAALLVKS

SEQ ID NO: 35 - protein, 9 aa, Artificial Sequence
  LV linker
PALAALVKS

SEQ ID NO: 36 - protein, 931 aa, Artificial Sequence
  L4-L TEN
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQDTGQLLKIAKRGGVTAVEAVHAWRNALTGAPLNLTPAQVVAIASNNGGKQALETVQRLLPVLCQAHGLTPDQVVAIASHDGGKQALETVQRLLPVLCQDHGLTPAQVVAIASNIGGKQALETVQRLLVLCQAHGLTPAQVVAIASNGGGKQALETVQRLLPVLCQDHGLTPEQVVAIASHDGGKQALETVQRLLPVLCQAHGLTPAQVVAIASNIGGKQALETVQRLLPVLCQDHGLTPEQVVAIASNIGGKQAETVQRLLPVLCQAHGLTPAQVVAIASHDGGKQALETVQRLLPVLCQAHGLTPAQVVAIASHDGGKQALETVQRLLPVLCQAHGLTPAQVVAIASHDGGKQALETVQRLLPVLCQDHGLTPDQVVAIAHDGGKQALETVQRLLPVLCQAHGLTPDQVVAIASNIGGKQALETVQRLLPVLCQAHGLTPAQVVAIASNGGGKQALETVQRLLPVLCQDHGLTPEQVVAIASHDGGKQALETVQRLLPVLCQAHGLTAQVVAIASNIGGKQALETVQRLLPVLCQDHGLTPAQVVAIASNGGGKQALETVQRLLPVLCQAHGLTPEQVVAIASNGGGKQALESIVAQLSRPDPALAALLVKSELEEKKSELRHKLKYVPHEYIEIEIARNPTQDRILEMKVMEFFMKVYGYRGEHLGGSRKPDGAIYTVGSPIDYGVIVDTKAYSGGYNLPIGQADAMQSYVEENQTRNKHINPNEWWKVYPSSVTEFKFLFVSGHFKGNYKAQLTRLNHINCNGAVLSVEELLIGGEMIKAGTLTLEEVRRKFNNGEINFLD

SEQ ID NO: 37 - DNA, 2796 bp, Artificial Sequence
  L4-L TEN
atggtgtacccctacgacgtgcccgactacgccgaattgcctccaaaaaagaagagaaaggtagggatccgaattcaagatctacgcacgctcggctacagccagcagcaacaggagaagatcaaacgaaggttcgttcgacagtggcgcagcaccacgaggcactggtcggccatgggtttacacacgcgcacatcgttgcgctcagccaacacccggcagcgttagggaccgtcgctgtcaagtatcaggactgatcgcagcgttgccagaggcgacacacgaagcgatcgttggcgtcggcaaacagtggtccggcgcacgcgctctggaggccttgctcacggtggcgggagagttgagaggtccaccgttacagttgacacaggccaacttctcaagattgcaaaacgtggcggcgtgaccgcagtggaggcagtgcatgcatggcgcaatgcactgacgggtgcccccctgaacctgacccctgctcaggttgtcgcaattgtagcaacaatggaggaaaacaagctttggagacagttcagaggttgttgcccgtcctctgtcaggctcatggcttgactcctgaccaggtggtcgctattgctagtcacgacggcggtaaacaagcctcgaaacagtgcagagacttcttcctgttctgtgccaagaccatggtcttacaccagctcaggtcgttgccatcgcctctaatattggtggaaaacaggcactcgagactgtgcaaaggcttctgccgtcctttgccaagcacatgggttgactcccgctcaggtggtggctattgcaagtaatggaggagggaagcaggccttggagacagtccaacgcttgctgcccgttctttgtcaggatcatgggttgaacccgagcaagttgtggcaattgcttcacacgatggtggcaaacaggctttggaaacagttcaaagattgttgcctgtcctttgccaagctcatggacttactccagcacaggtggtggccatcgcaccaacataggaggtaaacaagcactggaaaccgtccagaggcttttgcctgtcctctgccaggatcacggtctgacaccagagcaggtggtcgccatcgcatccaatattggtggaaaacaagctctgaaactgtccagagacttttgccagtgctctgtcaagctcatggcctcactcctgctcaggttgtggccattgccagccacgatgggggtaagcaagcacttgaaacagttcaaagactgcttcccgtgctttgtcaggcacacgggctgactcccgcacaagtcgtcgccatcgcctcacatgacggaggcaacaagcactggagacagttcaacgcctcctccctgtcttgtgccaagctcatgggctgacccctgcccaggtggtggccattgcctcccacgatggaggtaagcaggctctggagacagttcaaagatgcttccagttctttgccaggatcacggtttgacacccgaccaagtcgttgcaatcgccagtcatgatggtggtaagcaagcactcgaaaccgtccagcgcttgctgcccgtgctctgccaggctcagggctgacacctgatcaggtcgtggccatcgcatcaaatatagggggtaaacaagctttggagactgtccagaggctcctccccgtcttgtgtcaagcccatggactgactcccgctcaggtggtggtattgcaagtaatggaggagggaagcaggccttggagacagtccaacgcttgctgcccgttctttgtcaggatcatgggttgacacccgagcaagttgtggcaattgcttcacacgatggtggcaaaaggctttggaaacagttcaaagattgttgcctgtcctttgccaagctcatggacttactccagcacaggtggtggccatcgcatccaacataggaggtaaacaagcactggaaaccgtccagaggctttgcctgtcctctgccaggatcatggtctgactccagcccaagttgtcgccattgccagtaatggtggtggtaagcaggccctggagactgtgcaaaggcttctgccagttttgtgccaagcacacgtctgactccggaacaggtggtggcgattgcaagcaacggcggcggcaaacaggctctagagagcattgttgcccagctctccagacctgatccggcgctagccgcgttgctagtcaaaagtgaactcaggagaagaaatctgaacttcgtcataaattgaaatatgtgcctcatgaatatattgaattaattgaaattgccagaaatcccactcaggatagaattcttgaaatgaaggtaatggaattttttataaagtttatggatatagaggtgagcatttgggtggatcaaggaaaccggacggagcaatttatactgtcggatctcctattgattacggtgtgatcgtggatactaaagcttatagcggaggttatatctgccaattggccaagcagatgccatgcaaagctatgtcgaagaaaatcaaacacgaaacaaacatatcaaccctaatgaatggtggaaagtctatccatcttctgtaacggaatttaagtttttattgtgagtggtcactttaaaggaaactacaaagctcagcttacacgattaaatcatatcactaattgtaatggagctgttcttagtgtagaagagcttttaattggtggagaaatgattaaagccggacattaaccttagaggaagtgagacggaaatttaataacggcgagataaactttctcgattag

SEQ ID NO: 38 - protein, 999 aa, Artificial Sequence
  L4-R TEN
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQDTGQLLKIAKRGGVTAVEAVHAWRNALTGAPLNLTPAQVVAIASNIGGKQALETVQRLLPVLCQAHGLTPEQVVAIASNNGGKQALETVQRLLPVLCQAHGLTPDQVVAIASNGGGKQALETVQRLLVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPAQVVAIASNGGGKQAETVQRLLPVLCQAHGLTPAQVVAIASNNGGKQALETVQRLLPVLCQDHGLTPAQVVAIASNIGGKQALETVQRLLPVLCQAHGLTPEQVVAIASNIGGKQALETVQRLLPVLCQAHGLTPDQVVAIAHDGGKQALETVQRLLPVLCQAHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQDHGLTDQVVAIASNGGGKQALETVQRLLPVLCQAHGLTPAQVVAIASHDGGKQALETVQRLLPVLCQAHGLTPDQVVAIASHDGGKQALETVQRLLPVLCQDHGLTPDQVVAIASHDGGKQALETVQRLLPVCQDHGLTPEQVVAIASNGGGKQALESIVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKASGGYNLPIGQAREMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 39 - DNA, 2871 bp, Artificial Sequence
  L4-R TEN
gaaggttcgttcgacagtggcgcagcaccacgaggcactggtcggccatgggtttacacacgcgcacatcgttgcgctcagccaacacccggcagcgttagggaccgtcgctgtcaagtatcaggactgatcgcagcgttgccagaggcgacacacgaagcgatcgttggcgtcggcaaacagtggtccggcgcacgcgctctggaggccttgctcacggtggcgggagagttgagaggtccaccgttacagttgacacaggccaacttctcaagattgcaaaacgtggcggcgtgaccgcagtggaggcagtgcatgcatggcgcaatgcactgacgggtgcccccctgaaccttactcctgctcaggtcgtcgccattgatctaatatcgggggcaagcaagctcttgagactgtccaaagactgctgccagtgctgtgccaagcccacggcctcacaccagagcaggtggtggccatcgccagtaacaatggcggtaaacaagcctggaaactgttcagaggctccttccagttctgtgccaggcccatggacttaccccagatcaagttgttgctattgctagcaacgggggcggaaaacaggctctcgaaacagttcagcgcctgttgccgtgttgtgtcaggatcatgggttgacacctgaccaagtcgttgcaatcgcttcaaacggtggaggtaaacaagctttggaaaccgtccaacgccttcttccagttctttgtcaggatcatggtcttaacctgagcaggtggttgcaattgccagcaatggtggaggcaaacaagctctggagacagtgcagagacttttgcctgtcctttgccaggcccacggattgaccccagaccaggttgtcgctattgcacacatgacggtggcaagcaagctctcgaaactgtccagagattgctccctgtcttgtgtcaagcacatggtttgacaccagcacaggtggttgcaattgcttcaaacggaggtggaaaacaagcattgagacagtccagagacttcttcctgtgctttgtcaggctcacggactgactcccgctcaggtcgttgctatcgctagtaacaatggcggcaagcaggcactggaaactgttcagcgcctcctcccagtctctgccaagatcacggtttgactcccgctcaagtggtcgccatcgcctccaacataggaggtaaacaggctttggaaaccgttcagagattgttgcctgttttgtgtcaagcacatggcttgaccctgagcaagtggttgccattgccagtaatatcggcggcaagcaggctttggaaactgttcagagattgctgcccgttctttgccaagcacatggcttgacacccgatcaagttgttgctatcgctagcatgatggagggaaacaagcccttgagactgtgcaacggctgcttccagtgttgtgccaagctcatggacttactcccgatcaggtcgtggctattgcatcaaatggtggtggcaaacaagcactggaaccgttcaaaggttgcttcctgttctgtgtcaggaccacggactgactcctgagcaggttgtcgctatcgcttccaatggcggtggcaaacaggcattggagacagtccaaagactcttgcccgtctgtgtcaggcacacgggcttacaccagatcaggtggtcgccatcgccagtcatgacggcggaaaacaggcactggagactgtgcaacgcttgcttcctgttctttgtcaagatcacggcttgactccgaccaggtcgtggccatcgcctcaaatgggggagggaagcaagcacttgaaactgttcaacggcttctcccagtgctgtgtcaggctcatgggctcaccccagctcaagtcgtcgctatcgctagtctgatggggggaaacaggctctcgaaactgtgcagaggctgctccccgtgctttgtcaggctcacggtttgacccccgaccaggtcgttgcaatcgcctctcatgacggcggcaagcaagccctcgagctgtgcaaaggctgcttcccgtcttgtgccaagatcatggcctcactcctgatcaggtggtggccattgcttcacacgatgggggcaagcaggctcttgaaaccgttcagagacttttgccagtccttgtcaggaccacggtctgactccggaacaggtggtggcgattgcaagcaacggcggcggcaaacaggctctagagagcattgttgcccagctctccagacctgatccggcgctagccgcgttgctagcaaaagtgaactcgaggagaagaaatctgaacttcgtcataaattgaaatatgtgcctcatgaatatattgaattaattgaaattgccagaaatcccactcaggatagaattcttgaaatgaaggtatggaattttttatgaaagtttatggatatagaggtgagcatttgggtggatcaaggaaaccggacggagcaatttatactgtcggatctcctattgattacggtgtgatcgtggatactaaagcttaagcggaggttataatctgccaattggccaagcacgagaaatgcaacgatatgtcgaagaaaatcaaacacgaaacaaacatatcaaccctaatgaatggtggaaagtctatccatcttctgtaacggatttaagtttttatttgtgagtggtcactttaaaggaaactacaaagctcagcttacacgattaaatcatatcactaattgtaatggagctgttcttagtgtagaagagcttttaattggtggagaatgattaaagccggcacattaaccttagaggaagtgagacggaaatttaataacggcgagataaactttctcgattag

SEQ ID NO: 40 - protein, 34 aa, Artificial Sequence
  HD module
LTPEQVVAIASHDGGKQALETVQRLLPVLCQDHG

SEQ ID NO: 41 - protein, 34 aa, Artificial Sequence
  HD module
LTPAQVVAIASHDGGKQALETVQRLLPVLCQAHG

SEQ ID NO: 42 - protein, 34 aa, Artificial Sequence; identical to SEQ ID NO: 40
  HD module

SEQ ID NO: 43 - protein, 34 aa, Artificial Sequence
  HD module
LTPDQVVAIASHDGGKQALETVQRLLPVLCQAHG

SEQ ID NO: 44 - protein, 34 aa, Artificial Sequence
  HD module
LTPDQVVAIASHDGGKQALETVQRLLPVLCQDHG

SEQ ID NO: 45 - protein, 34 aa, Artificial Sequence
  NG module
LTPEQVVAIASNGGGKQALETVQRLLPVLCQDHG

SEQ ID NO: 46 - protein, 34 aa, Artificial Sequence
  NG module
LTPAQVVAIASNGGGKQALETVQRLLPVLCQAHG

SEQ ID NO: 47 - protein, 34 aa, Artificial Sequence
  NG module
LTPAQVVAIASNGGGKQALETVQRLLPVLCQDHG

SEQ ID NO: 48 - protein, 34 aa, Artificial Sequence
  NG module
LTPDQVVAIASNGGGKQALETVQRLLPVLCQAHG

SEQ ID NO: 49 - protein, 34 aa, Artificial Sequence
  NG module
LTPDQVVAIASNGGGKQALETVQRLLPVLCQDHG

SEQ ID NO: 50 - protein, 34 aa, Artificial Sequence
  NI module
LTPEQVVAIASNIGGKQALETVQRLLPVLCQDHG

SEQ ID NO: 51 - protein, 34 aa, Artificial Sequence
  NI module
LTPAQVVAIASNIGGKQALETVQRLLPVLCQAHG

SEQ ID NO: 52 - protein, 34 aa, Artificial Sequence
  NI module
LTPAQVVAIASNIGGKQALETVQRLLPVLCQDHG

SEQ ID NO: 53 - protein, 34 aa, Artificial Sequence
  NI module
LTPDQVVAIASNIGGKQALETVQRLLPVLCQAHG

SEQ ID NO: 54 - protein, 34 aa, Artificial Sequence
  NI module
LTPDQVVAIASNIGGKQALETVQRLLPVLCQDHG

SEQ ID NO: 55 - protein, 34 aa, Artificial Sequence
  NN module
LTPEQVVAIASNNGGKQALETVQRLLPVLCQDHG

SEQ ID NO: 56 - protein, 34 aa, Artificial Sequence
  NN module
LTPAQVVAIASNNGGKQALETVQRLLPVLCQAHG

SEQ ID NO: 57 - protein, 34 aa, Artificial Sequence; identical to SEQ ID NO: 55
  NN module

SEQ ID NO: 58 - protein, 34 aa, Artificial Sequence
  NN module
LTPDQVVAIASNNGGKQALETVQRLLPVLCQAHG

SEQ ID NO: 59 - protein, 34 aa, Artificial Sequence
  NN module
LTPDQVVAIASNNGGKQALETVQRLLPVLCQDHG

SEQ ID NO: 60 - protein, 2 aa, Artificial Sequence
  L2 Linker
SI

SEQ ID NO: 61 - protein, 5 aa, Artificial Sequence
  L3 Linker
SIVAQ

SEQ ID NO: 62 - protein, 16 aa, Artificial Sequence
  L4 Linker
SIVAQLSRPDPALAAL

SEQ ID NO: 63 - protein, 198 aa, Artificial Sequence
  FokI nuclease domain
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 64 - DNA, 20 bp, Artificial Sequence
  Synthetic
tgggggaggtggcgaggaac

SEQ ID NO: 65 - DNA, 18 bp, Artificial Sequence
  Synthetic
tgcatcaaccccatcatc

SEQ ID NO: 66 - DNA, 20 bp, Artificial Sequence
  Synthetic
tagtttctgaacttctcccc

SEQ ID NO: 67 - DNA, 18 bp, Artificial Sequence
  Synthetic
tgcatcaatcccatcatc

SEQ ID NO: 68 - DNA, 20 bp, Artificial Sequence
  Synthetic
taccttctgaacttctcccc

SEQ ID NO: 69 - DNA, 18 bp, Artificial Sequence
  Synthetic
tgcctgaatcctctcatc

SEQ ID NO: 70 - DNA, 20 bp, Artificial Sequence
  Synthetic
tatcttctgaacttctcccc

SEQ ID NO: 71 - DNA, 18 bp, Artificial Sequence
  Synthetic
tgccttaatcccatcatc

SEQ ID NO: 72 - DNA, 20 bp, Artificial Sequence
  Synthetic
tacttgcgaaatttctcccc

SEQ ID NO: 73 - DNA, 18 bp, Artificial Sequence
  Synthetic
tgcctaaaccccctcatc

SEQ ID NO: 74 - DNA, 20 bp, Artificial Sequence
  Synthetic
ttgtccctgaaggtctcccc

SEQ ID NO: 75 - DNA, 18 bp, Artificial Sequence
  Synthetic
tgcatgaacccggtgatc

SEQ ID NO: 76 - DNA, 20 bp, Artificial Sequence
  Synthetic
tacttccggaacctctctcc

SEQ ID NO: 77 - DNA, 18 bp, Artificial Sequence
  Synthetic
ttctttaaccccattagc

SEQ ID NO: 78 - DNA, 18 bp, Artificial Sequence
  Synthetic
aacatcaacccctccatc

SEQ ID NO: 79 - DNA, 18 bp, Artificial Sequence
  Synthetic
tggagcaatgccattatc

SEQ ID NO: 80 - DNA, 18 bp, Artificial Sequence
  Synthetic
tgcatccaacctttcatc

SEQ ID NO: 81 - DNA, 18 bp, Artificial Sequence
  Synthetic
tgtgtcaacccagtgatc

SEQ ID NO: 82 - DNA, 20 bp, Artificial Sequence
  Synthetic
tacttccggaacctctcacc

SEQ ID NO: 83 - DNA, 18 bp, Artificial Sequence
  Synthetic
ttcagtatccccatcagc

SEQ ID NO: 84 - DNA, 20 bp, Artificial Sequence
  Synthetic
gagtttctgtgcttctcagc

SEQ ID NO: 85 - DNA, 18 bp, Artificial Sequence
  Synthetic
ttcattaatcccctcata

SEQ ID NO: 86 - DNA, 18 bp, Artificial Sequence
  Synthetic
agcctcaacttcctcatc

SEQ ID NO: 87 - DNA, 55 bp, Artificial Sequence
  Synthetic
gcaacatgctggtcatcctcatcctgataaactgcaaaaggctgaagagcatgac

SEQ ID NO: 88 - DNA, 55 bp, Artificial Sequence
  Synthetic
gtcatgctcttcagccttttgcagtttatcaggatgaggatgaccagcatgttgc

SEQ ID NO: 89 - DNA, 18 bp, Artificial Sequence
  Synthetic
tgctggtcatcctcatcc

SEQ ID NO: 90 - DNA, 17 bp, Artificial Sequence
  Synthetic
tgctggtcatcctcatc

SEQ ID NO: 91 - DNA, 16 bp, Artificial Sequence
  Synthetic
tgctggtcatcctcat

SEQ ID NO: 92 - DNA, 15 bp, Artificial Sequence
  Synthetic
tgctggtcatcctca

SEQ ID NO: 93 - DNA, 14 bp, Artificial Sequence
  Synthetic
tgctggtcatcctc

SEQ ID NO: 94 - DNA, 13 bp, Artificial Sequence
  Synthetic
tgctggtcatcct

SEQ ID NO: 95 - DNA, 12 bp, Artificial Sequence
  Synthetic
tgctggtcatcc

SEQ ID NO: 96 - DNA, 11 bp, Artificial Sequence
  Synthetic
tgctggtcatc

SEQ ID NO: 97 - protein, 11 aa, Artificial Sequence
  Synthetic
LAALTNDHLVA

SEQ ID NO: 98 - protein, 8 aa, Artificial Sequence
  Synthetic
QLVKSELE

SEQ ID NO: 99 - DNA, 49 bp, Artificial Sequence
  Synthetic
ttgtgggcaacatgctggtcatcctcatcctgataaactgcaaaaggct

SEQ ID NO: 100 - DNA, 48 bp, Artificial Sequence
  Synthetic
ttgtgggcaacatgctggtcatcctcatctgataaactgcaaaaggct

SEQ ID NO: 101 - DNA, 48 bp, Artificial Sequence
  Synthetic
ttgtgggcaacatgctggtcatcctcatcctgaaaactgcaaaaggct

SEQ ID NO: 102 - DNA, 47 bp, Artificial Sequence
  Synthetic
ttgtgggcaacatgctggtcatcctcatcctgaaactgcaaaaggct

SEQ ID NO: 103 - DNA, 45 bp, Artificial Sequence
  Synthetic
ttgtgggcaacatgctggtcatcctcatccaaactgcaaaaggct

SEQ ID NO: 104 - DNA, 49 bp, Artificial Sequence; identical to SEQ ID NO: 99
  Synthetic

SEQ ID NO: 105 - DNA, 54 bp, Artificial Sequence
  Synthetic
ttgtgggcaacatgctggtcatcctcatcctgatctgataaactgcaaaaggct

SEQ ID NO: 106 - DNA, 75 bp, Artificial Sequence
  Synthetic
atgacgcactgctgcatcaaccccatcatctatgcctttgtcggggagaagttcagaaactacctcttagtcttc

SEQ ID NO: 107 - DNA, 75 bp, Artificial Sequence
  Synthetic
gaagactaagaggtagtttctgaacttctccccgacaaaggcatagatgatggggttgatgcagcagtgcgtcat

SEQ ID NO: 108 - DNA, 16 bp, Artificial Sequence
  Synthetic
tgcatcaaccccatca

SEQ ID NO: 109 - DNA, 20 bp, Artificial Sequence
  Synthetic
cccctcttcaagtctttgat

SEQ ID NO: 110 - DNA, 17 bp, Artificial Sequence
  Synthetic
tgcatcaaccccatcat

SEQ ID NO: 111 - DNA, 19 bp, Artificial Sequence
  Synthetic
ccctcttcaagtctttgat

SEQ ID NO: 112 - DNA, 18 bp, Artificial Sequence
  Synthetic
tgcatcaaccccatcatc

SEQ ID NO: 113 - DNA, 18 bp, Artificial Sequence
  Synthetic
cctcttcaagtctttgat

SEQ ID NO: 114 - DNA, 19 bp, Artificial Sequence
  Synthetic
tgcatcaaccccatcatct

SEQ ID NO: 115 - DNA, 17 bp, Artificial Sequence
  Synthetic
ctcttcaagtctttgat

SEQ ID NO: 116 - DNA, 64 bp, Artificial Sequence
  Synthetic
gacgcactgctgcatcaaccccatcatctatgcctttgtcggggagaagttcagaaactacctc

SEQ ID NO: 117 - DNA, 63 bp, Artificial Sequence
  Synthetic
gacgcactgctgcatcaaccccatcatctatgctttgtcggggagaagttcagaaactacctc

SEQ ID NO: 118 - DNA, 62 bp, Artificial Sequence
  Synthetic
gacgcactgctgcatcaaccccatcatctatgtttgtcggggagaagttcagaaactacctc

SEQ ID NO: 119 - DNA, 61 bp, Artificial Sequence
  Synthetic
gacgcactgctgcatcaaccccatcatctatgccgtcggggagaagttcagaaactacctc

SEQ ID NO: 120 - DNA, 54 bp, Artificial Sequence
  Synthetic
gacgcactgctgcatcaaccccatcatgtcggggagaagttcagaaactacctc

SEQ ID NO: 121 - DNA, 42 bp, Artificial Sequence
  Synthetic
gacgcactgctgcatgtcggggagaagttcagaaactacctc

SEQ ID NO: 122 - DNA, 64 bp, Artificial Sequence; identical to SEQ ID NO: 116
  Synthetic

SEQ ID NO: 123 - DNA, 67 bp, Artificial Sequence
  Synthetic
gacgcactgctgcatcaaccccatcatctatgcctcctttgtcggggagaagttcagaaactacctc

SEQ ID NO: 124 - DNA, 64 bp, Artificial Sequence; identical to SEQ ID NO: 116
  Synthetic

SEQ ID NO: 125 - DNA, 63 bp, Artificial Sequence
  Synthetic
gacgcactgctgcatcaaccccatcatctatgcctagtcggggagaagttcagaaactacctc

SEQ ID NO: 126 - DNA, 56 bp, Artificial Sequence
  Synthetic
tgctgcatcaagcccatcatctatgcctttgtcggggagaagttcagaaactacct

SEQ ID NO: 127 - DNA, 56 bp, Artificial Sequence
  Synthetic
aggtagtttctgaacttctccccgacaaaggcatagatgatgggcttgatgcagca

SEQ ID NO: 128 - DNA, 56 bp, Artificial Sequence
  Synthetic
tgctgcatcaatcccatcatctatgccttcgttggggagaagttcagaaggtatct

SEQ ID NO: 129 - DNA, 56 bp, Artificial Sequence
  Synthetic
agataccttctgaacttctccccaacgaaggcatagatgatgggattgatgcagca

SEQ ID NO: 130 - DNA, 59 bp, Artificial Sequence
  Synthetic
tttggttttgtgggcaacatgctggtcatcctcatcctgataaactgcaaaaggctgaa

SEQ ID NO: 131 - DNA, 59 bp, Artificial Sequence
  Synthetic
aaaccaaaacacccgttgtacgaccagtaggagtaggactatttgacgttttccgactt

SEQ ID NO: 132 - DNA, 59 bp, Artificial Sequence
  Synthetic
tttggttttgtgggcaacatgctggtcgtcctcatcttaataaactgcaaaaagctgaa

SEQ ID NO: 133 - DNA, 59 bp, Artificial Sequence
  Synthetic
aaaccaaaacacccgttgtacgaccagcaggagtagaattatttgacgtttttcgactt

SEQ ID NO: 134 - DNA, 92 bp, Artificial Sequence
  Synthetic
tgggcaacatgctggtcgtcctcatcttaataaactgcaaaaagcttgggcaacatgctggtcatcctcatcctgataaactgcaaaaggct

SEQ ID NO: 135 - DNA, 46 bp, Artificial Sequence
  Synthetic
tgggcaacatgctggtcgtcctcatcttaataaactgcaaaaggct

SEQ ID NO: 136 - DNA, 44 bp, Artificial Sequence
  Synthetic
tgggcaacatgctggtcgtcctcatctttaaactgcaaaaggct

SEQ ID NO: 137 - DNA, 46 bp, Artificial Sequence
  Synthetic
tgggcaacatgctggtcgtcctcatcctgataaactgcaaaaggct

SEQ ID NO: 138 - DNA, 37 bp, Artificial Sequence
  Synthetic
tgggcaacatgctggtcgtcctcatctgcaaaaggct

SEQ ID NO: 139 - DNA, 23 bp, Artificial Sequence
  Synthetic
tgggcaacatgctgcaaaaggct

SEQ ID NO: 140 - DNA, 20 bp, Artificial Sequence
  Synthetic
ggggagaagttcagaaacta

SEQ ID NO: 141 - protein, 7 aa, Artificial Sequence
  Synthetic
GGKQALE

SEQ ID NO: 142 - protein, 8 aa, Artificial Sequence
  Synthetic
QLVKSELE

SEQ ID NO: 143 - protein, 9 aa, Artificial Sequence
  Synthetic
GGKQALESI

SEQ ID NO: 144 - protein, 12 aa, Artificial Sequence
  Synthetic
GGKQALESIVAQ

SEQ ID NO: 145 - protein, 23 aa, Artificial Sequence
  Synthetic
GGKQALESIVAQLSRPDPALAAL

SEQ ID NO: 146 - protein, 7 aa, Artificial Sequence
  Synthetic
LVKSELE

SEQ ID NO: 147 - protein, 37 aa, Artificial Sequence
  Synthetic
GGRPALESIVAQLSRPDPALAALTNDHLVALACLGGS

SEQ ID NO: 148 - protein, 24 aa, Artificial Sequence
  Synthetic
GGRPALESIVAQLSRPDPALAALT

SEQ ID NO: 149 - protein, 5 aa, Artificial Sequence
  Synthetic
RVAGS

(19) United States
(12) Patent Application Publication
(10) Pub. No.: US 2013/0217131 A1
(43) Pub. Date: Aug. 22, 2013

(54) GENOME ENGINEERING VIA DESIGNED TAL EFFECTOR NUCLEASES

(71) Applicants: Jin Soo Kim, Seoul (KR); Hye Joo Kim, Daejeon (KR)

(72) Inventors: Jin Soo Kim, Seoul (KR); Hye Joo Kim, Daejeon (KR)

(73) Assignee: TOOLGEN INCORPORATION, Seoul (KR)

(21) Appl. No.: 13/768,798

(22) Filed: Feb. 15, 2013

Related U.S. Application Data
(63) Continuation-in-part of application No. PCT/KR2012/000042, filed on Jan. 3, 2012.
(60) Provisional application No. 61/429,346, filed on Jan. 3, 2011.

(51) Int. Cl. C12N 9/22; C12N 15/90

(52) U.S. Cl. CPC C12N 9/22; C12N 15/902
USPC 435/462; 435/199; 536/23.2; 435/252.33; 435/254.2; 435/254.11; 435/258.1; 435/348; 435/419; 435/358; 435/367; 435/369; 435/365; 435/325; 435/366; 435/468; 435/471

(57) ABSTRACT

The present invention relates to a fusion protein having a TAL (transcription activator-like) effector (TALE) domain and a nucleotide cleavage domain, and more particularly, to the TAL effector nuclease comprising a TAL (transcription activator-like) effector (TALE) domain and a nucleotide cleavage domain, wherein the TALE domain includes one or more TALE-repeat modules, each of the TALE-repeat modules recognizing a single specific nucleic acid, and a use thereof.

The present application is a continuation-in-part of International Application No. PCT/KR2012/000042, filed Jan. 3, 2012, which claims priority to U.S. Provisional Patent Application No. 61/429,346, filed Jan. 3, 2011, the disclosures of which are herein incorporated by reference in their entireties.

### TECHNICAL FIELD

The present invention relates to a fusion protein having a TAL (transcription activator-like) effector (TALE) domain and a nucleotide cleavage domain (hereinafter referred to as “TAL effector nuclease”), and more particularly, to the TAL effector nuclease comprising a TAL (transcription activator-like) effector (TALE) domain and a nucleotide cleavage domain, wherein the TALE domain includes one or more TALE-repeat modules, each of the TALE-repeat modules specifically recognizing a single nucleic acid, and a use thereof.

### BACKGROUND

Genome engineering that allows targeted mutagenesis and gene correction in higher eukaryotic cells and organisms can be applied to a broad field of research, biotechnology, and molecular medicine. Zinc finger nucleases (hereinafter, referred to as “ZFN”s) are powerful and versatile tools for genome engineering that induce site-specific DNA double strand breaks (hereinafter, referred to as “DSB”s) in the genome, which in turn get repaired via homologous recombination or non-homologous end-joining (hereinafter, referred to as “NHEJ”) giving rise to a gene correction, gene disruption, and gene addition as well as chromosomal rearrangements. However, it is technically challenging and highly time-consuming to make a fully functional ZFN. Also ZFNs involve sequence-bias towards GNN-repeat sites, which in turn disrupt a precise manipulation of the genome at the base pair level.

To be specific, ideal tools for genome engineering in higher eukaryotic cells and organisms should meet the following criteria: they must be readily reprogrammable and have little or no sequence-bias. Although ZFNs are widely used for a targeted genome modification in plants, animals, and cultured cells, they do not meet the above-specified criteria. ZFNs are artificial DNA-cleaving enzymes composed of tailor-made zinc-finger DNA-binding arrays and the FokI nuclease domain derived from Flavobacterium okeanokoites. ZFNs induce site-specific DNA double strand breaks (DSBs), whose repair via endogenous DNA repair systems give rise to targeted genome modifications. First, zinc finger-DNA interactions are highly sensitive to DNA sequence of the target site, and thus zinc finger arrays made by modular assembly often fail to bind to their designated target sites. Second, ZFNs have sequence bias toward guanine-rich sites such as GNN-repeat sequences. Zinc finger arrays consist of at least 3 tandem arrays of zinc finger modules, and each zinc finger recognizes a 3-base pair (bp) subsite. Therefore, up to 64 different zinc fingers, each corresponding to one of the 64 triplet bases, are required to assemble zinc finger arrays. Although many zinc fingers with exquisite specificities are now used to make ZFNs, the lack of reliable zinc fingers that recognize certain 3-bp subsites, especially CNN and ANN triplets, has been a serious limiting factor in the field of genomic engineering. Thus, ZFNs that recognize target sites composed of these triplets may not be produced.

Recent findings of the factors that affect protein-DNA interactions of plant pathogen-derived TAL effectors (hereinafter, referred to as “TALE”s) may provide a new promising lead for development of powerful tools that overcome the above limitations. Unlike zinc fingers which recognize 3-bp subsites, each repeat module of TALEs interacts with a single base. Since there are at least four different repeat modules, each preferentially recognizing one of the four bases, it is possible to design TALEs (hereinafter, referred to as “dTALE”s) that specifically bind to the predetermined target site.

In order to make functional TAL Effector Nucleases (hereinafter, referred to as “TALEN”s) with genome-editing activity, the following critical parameters must be considered: i) the minimal DNA-binding domain of TALEs, ii) the length of the spacer between the two half-sites that constitute a target site (FIGS. 1a and b), and iii) the linker or fusion junction that connects the FokI nuclease domain to dTALEs (FIG. 1c).

### DESCRIPTION

### Technical Problem

In light of the above essential components, a broad use of the TALEN technology in a targeted genome editing is limited by a lack of the method for synthesizing functional TALENs, that is convenient, rapid and publicly available method. Thus, the present inventors have tried to develop a highly efficient and easy-to-practice TALEN and found that the DNA-binding modules of TALEs derived from plant pathogens can substitute for zinc fingers to make TALENs and that TALENs induce bona-fide genome modifications at endogenous sites in cultured human cells. Unlike ZFNs, TALENs can be designed to recognize any form of DNA sequence with little or no bias toward the base. In addition, TALENs can recognize a longer DNA sequence than ZFNs, which may contribute to their reduced cellular toxicity and off-target effects compared to ZFNs. It is expected that TALENs can be used widely for a precise genomic modification in plants, animals, and cultured cells, including human stem cells, and may add a new dimension to genome engineering by allowing researchers to modify the target sites that were not amenable by using ZFNs.

### Technical Solution

It is an object of the present invention to provide a fusion protein having nuclease activity, comprising a TAL (transcription activator-like) effector (TALE) domain and a nucleotide cleavage domain, wherein the TALE domain includes one or more TALE-repeat modules, each of the TALE-repeat modules recognizing a single specific nucleic acid.

It is another object of the present invention to provide a nucleotide sequence encoding a nucleotide sequence, encoding the fusion protein.

It is still another object of the present invention to provide a kit for cleavage, replacement or modification of nucleotide sequences in a targeted region, comprising one or more pairs of the fusion proteins.

It is still another object of the present invention to provide a cell comprising the fusion protein.

It is still another object of the present invention to provide a method for deletion, duplication, inversion, replacement, insertion or rearrangement of genomic DNA, comprising the step of cleaving specific sites in a genome using one or more pair of the fusion proteins.

### Advantageous Effects

Unlike ZFNs, TALENs can be designed to recognize any DNA sequence with little or no bias toward any base. In addition, TALENs can recognize longer DNA sequences, which may contribute to their reduced cellular toxicity and off-target effects compared to ZFNs. It is expected that TALENs can be used broadly for precise genomic modifications in plants, animals, and cultured cells including human stem cells, and may add a new dimension to genome engineering by allowing researchers to target sites that are not amenable for modifications using ZFNs.

### DESCRIPTION OF DRAWINGS

FIG. 1 shows targeted genome modifications using TALEN/ZFN hybrid pairs. (a) Schematic of ZFN, ZFN/TALEN, and TALEN pairs. These site-specific endonucleases function as dimers. (b) The ZFN-215 target site in the human CCR5 gene. The half-site sequence recognized by the ZFN monomer (215R) is shown in bold italics. The half-site sequences recognized by TALENs (L9.5 to L16.5) are shown under the CCR5 sequence. Dashes indicate bases corresponding to spacers, and the number of base pairs in the spacers is shown. (c) Amino acid sequences in the linkers (or fusion junctions) that connect the TALE domain to the FokI domain. (d) Relative luciferase activities of cells in which TALEN/ZFN pairs were expressed. Values are compared to that of cells expressing I-SceI, an intron-encoded endonuclease derived from S. cerevisiae, which is used as a positive control. p-Values are calculated with the Student's t-test; (*) p<0.01 (empty vector vs. TALEN/ZFN), (**) p<0.05 (L11.5 vs. L20.5) (e) TALEN/ZFN-driven genomic mutations revealed by the T7E1 assay. ZFN-215 consists of 215R and 215L. The positions of uncut and cut DNA bands are indicated. The numbers at the bottom of the gel indicate mutation frequencies. (f) DNA sequences of indels induced at the CCR5 target site by a TALEN/ZFN pair. The recognition sequences of L20.5 TALEN and 215R ZFN are underlined. Dashes indicate deleted bases and bold lowercase letters indicate inserted bases. The number of occurrences is shown in parenthesis. wt, wild-type.

FIG. 2 shows a schematic of the construction of dTALEs. (a) The four TALE-repeat modules used for the construction of dTALEs. The amino acid sequence of a repeat module is shown. XX denotes hyper-variable amino-acids at positions 12 and 13, which determine the specificity of base recognition. These two resides are shown in the boxes that represent repeat modules. (b) is the stepwise construction of dTALEs. One plasmid was digested with XbaI and XhoI to yield a vector backbone and the other with NheI and XhoI to yield an insert segment. To create a plasmid encoding a two-repeat array, the insert segment was ligated with the vector backbone. The resulting plasmids were subjected to the next round of subcloning using the same sets of restriction enzymes. Finally, modularly-assembled repeat arrays were subcloned into an expression vector that encodes the Δ153 N-terminal domain of AvrBs3 at the N terminus and the Fokl nuclease domain at the C terminus to create TALEN expression vectors.

FIG. 3 shows the complete amino acid sequences of the CCR5-targeting TALENs. Underlined are the two hyper-variable amino-acid residues that determine the specificity of base-recognition. The TALE domain is shown in the box and the FokI nuclease domain is shown in bold. The HA tag and the nuclear localization signal (NLS) at the N terminus are indicated. (a) is T1L20.5. (b) is T2L16.5. (c) is T2R18.5.

FIG. 4 shows the minimal DNA-binding domain of AvrBs3 identified by a transcriptional repression assay in HEK293 cells. The plasmids that encode the wild-type AvrBs3 protein or its truncated forms were co-transfected into HEK293 cells with a luciferase reporter plasmid. The reporter plasmid carries the firefly luciferase gene under the control of a synthetic promoter that consists of the initiator element and the TATA-box-containing UPA20 element, the target site of AvrBs3. A set of five GAL4 binding sites was included upstream of the promoter, and the plasmid encoding GAL4-VP16 was co-transfectedwith the reporter plasmid and each of the AvrBs3-encoding plasmids. Proteins that were able to bind to the UPA20 element could inhibit the transcriptional activation of the reporter gene. As a negative control, we used the reporter plasmid that contains the adenovirus major late TATA-box instead of the UPA20 element. Luciferase activities were measured 2 days after co-transfection. A schematic of the promoter is shown above the luciferase data. WT, wild-type AvrBs3.

FIG. 5 shows targeted genome modifications using TALEN pairs. (a) is The Z891 target site in the CCR5 gene. The two half-site sequences recognized by Z891 are shown in bold italics. The half-site sequences recognized by TALENs are shown under the CCR5 sequence. (b) is the relative luciferase activities of cells in which each of the combinatorial TALEN pairs was expressed. p-Values are calculated with the Student's t-test; (*) p<0.05 (empty vector vs. TALEN pairs) (c) is TALEN pair-driven genomic mutations detected by T7E1. (d) is DNA sequences of indels induced by a TALEN pair. Symbols are as in FIG. 1.

FIG. 6 shows off-target effects and cellular toxicity of TALEN pairs. (a) is DNA sequences of the CCR5 on-target and CCR2 off-target sites. Non-conserved bases at the two sites are shown in lowercase letters. The half-site sequences recognized by R18.5 and L17.5 are underlined. The two half-site sequences recognized by Z891 are shown in bold italics. (b) is PCR products corresponding to the 15-kbp chromosomal deletions. (c) is a T7E1 assay showing off-target mutations at the CCR2 site induced by Z891 but not by TALEN pairs. (d) is a T7E1 assay comparing the stability of nuclease-driven mutations. The T7E1 assay was performed at days 3 and 9 after transfection of TALEN, TALEN/ZFN, and ZFN pairs.

FIG. 7 shows off-target effects of TALEN/ZFN pairs at the ZFN-215 site. (a) is DNA sequences of the CCR5 on-target and CCR2 off-target sites. Non-conserved bases at the two sites are shown in lowercase letters. The half-site sequence recognized by L20.5 is underlined. The half-site sequence recognized by 215R is shown in bold italics. (b) is PCR products corresponding to the 15-kbp chromosomal deletions. (c) is DNA sequences of PCR products corresponding to the 15-kbp chromosomal deletions induced by the TALEN/ZFN pair, L20.5/215R. Dashes indicate deleted bases. Non-conserved bases at the two sites are shown in lowercase letters. The number of occurrences is shown in parenthesis. wt, wild-type.

FIG. 8 shows the DNA sequence and amino acid sequence of an assembled TALEN pair.

FIG. 9 shows the optimization of a TALEN architecture. (a) is a schematic diagram of the RFP-GFP reporter-based assay for measuring the gene-editing activities of various TALEN constructs. (b) shows a TALEN target site and amino acid sequence of the fused junctions where the TALE array is linked to the FokI domain. (c) shows a comparison of gene-editing activity among different TALEN constructs. Reporter plasmids and TALEN plasmids were co-transfected into HEK 293 cells, and the number of GFP+ cells were counted via flow cytometry. S+28 and S+63 are the two prototypes of TALEN architecture previously reported by Miller et al. (a TALE nuclease architecture for efficient genome editing. Nat Biotechnol 29, 143-148 (2011)). Error bars represent SEM of at least triplicates of the experiment.

FIG. 10 is a schematic diagram of the assembly of TALEN plasmids.

FIG. 11a is a schematic diagram of Golden-Gate assembly of TALEN plasmids. A total of 424 TALE array plasmids (=64×6+16×2+4×2) (KanR) and 8 FokI plasmids (AmpR) are used. FIG. 11b shows the result of a high-throughput Golden-Gate cloning in 96-well plates. Six TALE array plasmids and one FokI plasmid are mixed in each well of the plate. BsaI releases the TALE arrays and allows an ordered assembly of six TALE arrays into the FokI plasmid. 11c shows the result of a pilot test of 15 TALENs using the T7E1 assay. Asterisks indicate the expected position of DNA bands representing the TALENs cleaved by T7E1. The numbers at the bottom of the gel indicate mutation frequencies measured by a band intensity.

FIG. 12 demonstrates targeted gene-disrupting activities of TALENs.

As one aspect of the invention, the present invention relates to a fusion protein having a nuclease activity, comprising a TAL (transcription activator-like) effector (TALE) domain and a nucleotide cleavage domain, wherein the TALE domain includes one or more TALE-repeat modules, each of the TALE-repeat modules recognizing a single nucleic acid.

The term “TAL (transcription activator-like) effector nuclease (TALEN)” of the present invention refers to a nuclease capable of recognizing and cleaving its target site. TALEN refers to a fusion protein comprising a TALE domain and a nucleotide cleavage domain. Preferably, the fusion protein may consist of the N-terminal domain, one or more of TALE-repeat modules followed by a half-repeat module, a linker, and a nucleotide cleavage domain. Preferably, the N-terminal domain may have an amino acid sequence of SEQ ID NO:28.

Preferably, the fusion protein may further comprise a HA tag and a Nuclear Localization Signal (NLS) sequence upstream of the N-terminal domain.

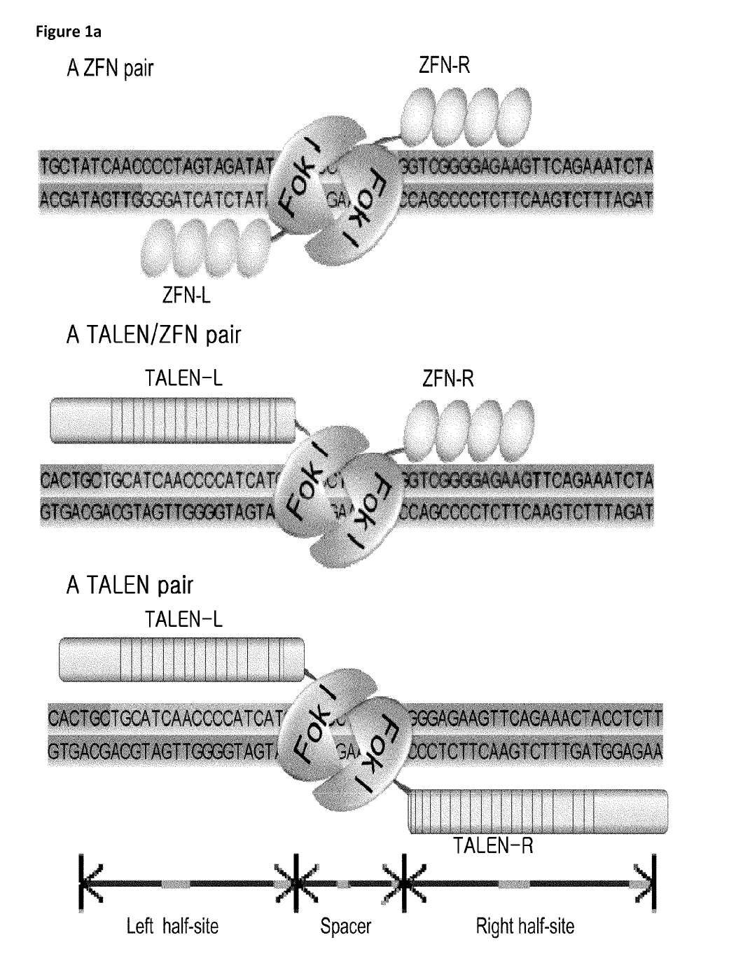

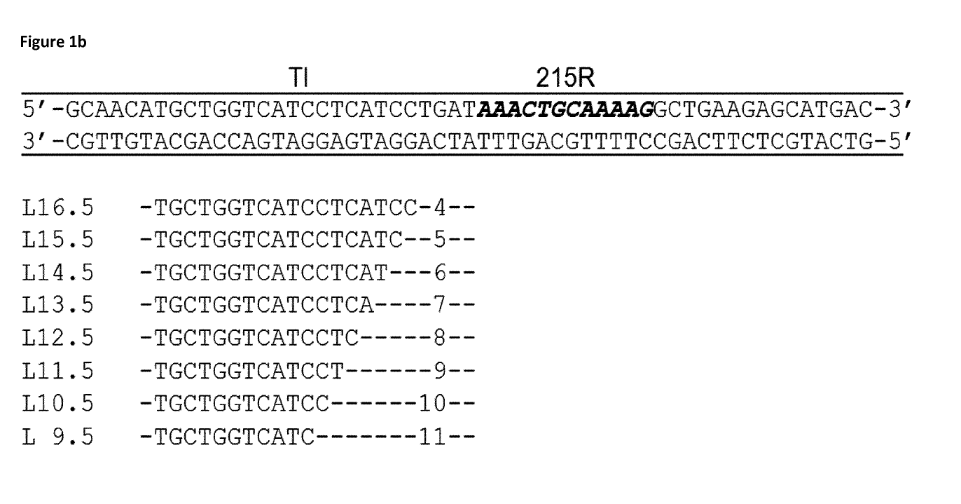

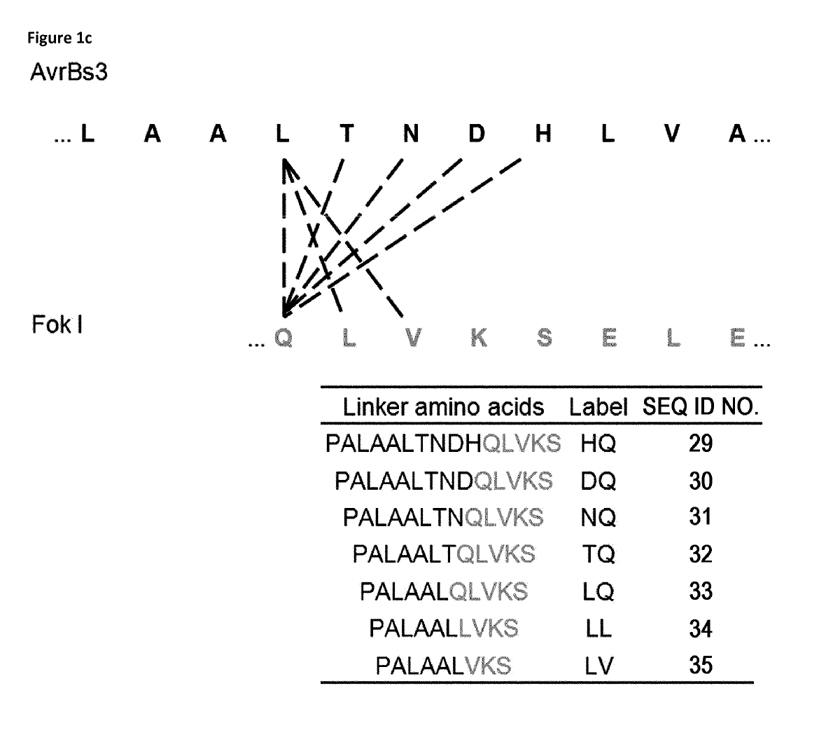

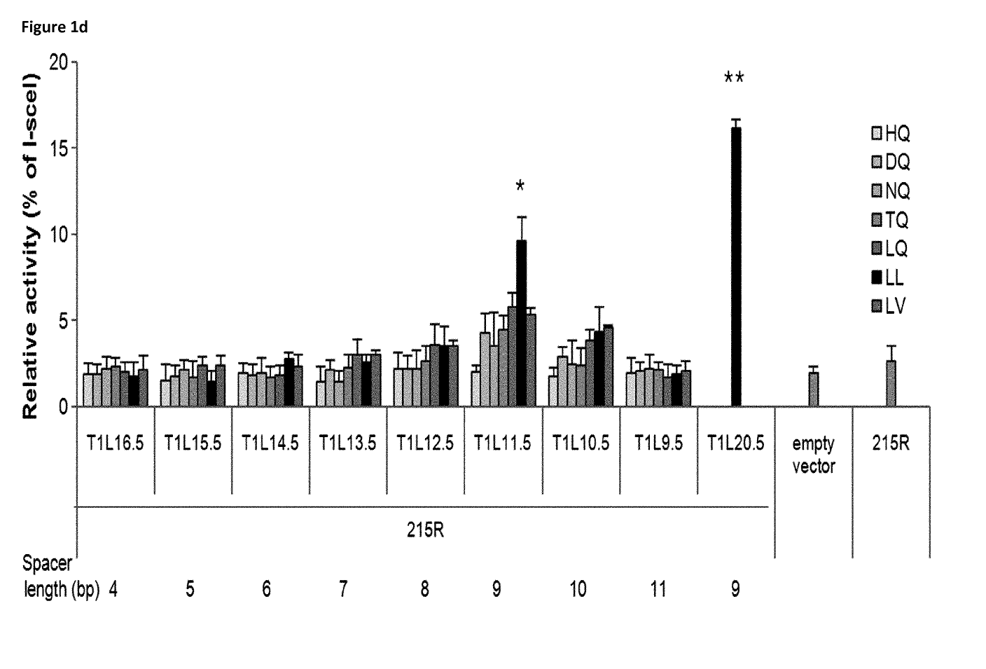

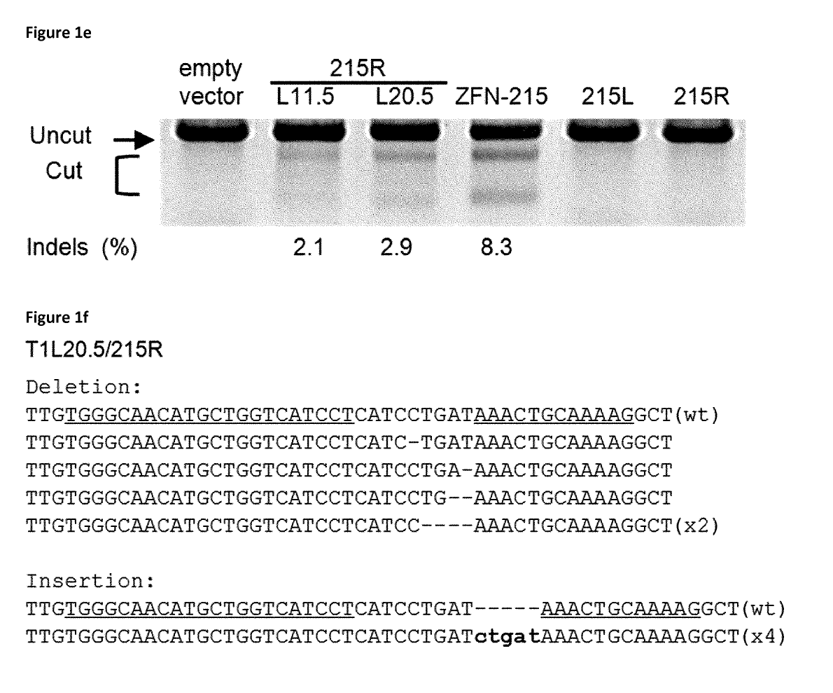

In the present invention, the terms “TAL effector nuclease” and “TALEN” can be used interchangeably. TAL effectors are the proteins secreted by Xanthomonas bacteria via type-III secretion system when they infect the plant species. These proteins can bind a promoter sequence in the host plant and activate the expression of the target plant gene that can promote bacterial infection. They recognize a DNA sequence of plant by a central repeat domain consisting of 1 to 34 amino acids. Therefore, TALEs were considered as a platform for developing a new promising tool for genomic engineering. However, until now, there has been a limitation in developing functional TALENs with a genome-editing activity since the following critical parameters were not known: i) the minimal DNA-binding domain of TALEs, ii) the length of the spacer between the two half-sites that constitute a target site (FIGS. 1a and b), and iii) the linker or fused junction that connects the FokI nuclease domain with dTALEs (FIG. 1c). The present inventors are the first to identify these parameters. The TALEN may have an amino acid sequence of SEQ ID NOs: 3, 6, 9, 36 or 38, but is not limited thereto.

In the present invention, the term “N-terminal domain” refers to a N-terminal of TALEN.

The TALE domain of the present invention refers to a protein domain that binds to a nucleotide in a sequence-specific manner through one or more TALE-repeat modules. The TALE domain comprises at least one of the TALE-repeat modules, preferably from one to thirty TALE-repeat modules, but it is not limited thereto. In the present invention, the terms “TAL effector domain” and “TALE domain” can be used interchangeably. The TALE domain may comprise a half-repeat module.

In the present invention, the term “the half-repeat module” refers to the last TALE repeat sequence of ˜20 amino acids in length that are found in naturally-occurring TAL effectors.

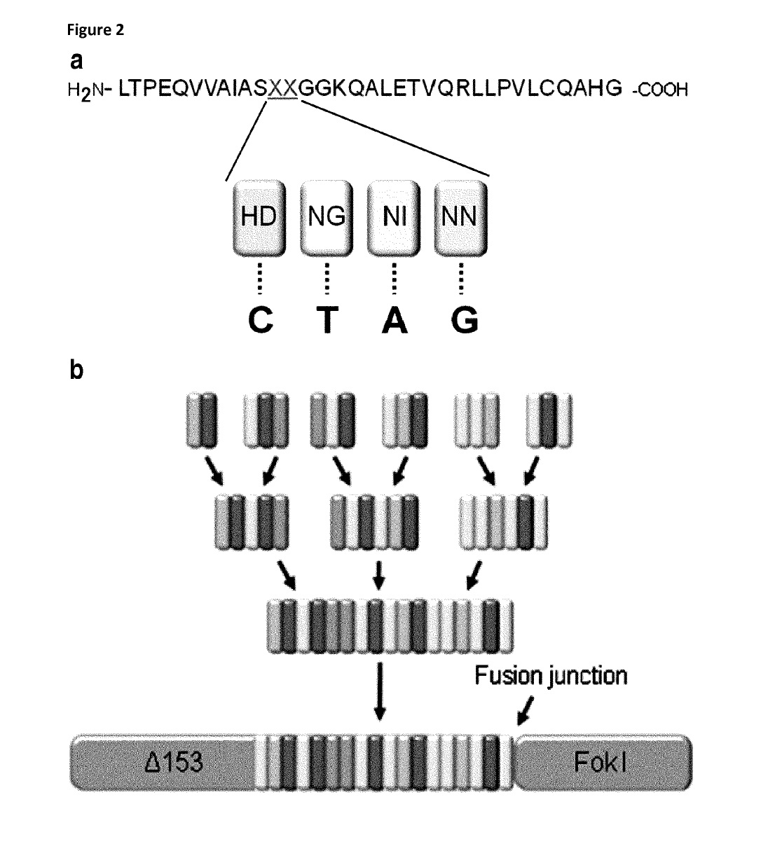

The TALE-repeat modules of the present invention refer to the binding domain of the amino acid sequence. The TALE-repeat modules of the present invention have the sequences identical to those of the naturally-occurring wild-type TALE-repeat modules or the sequences that are modified by substitution of amino acids in the wild-type sequence. The wild-type TALE-repeat module may be derived from any plant pathogen. Preferably, the TALE-repeat module of the present invention includes the amino acid sequence, represented by FIG. 2a. The TALE-repeat module may have the amino acid sequence of SEQ ID NOs: 24, 25, 26, 27, 40, 41, 42, 43, 44, 45, 46, 47, 48, 49, 50, 51, 52, 53, 54, 55, 56, 57, 58, or 59, but is not limited thereto.

TALE-repeat module may have the following general amino acid sequences:

H₂N-LTPE(or A or D)QVVAIASXXGGKQALETVQRLLPVLCQA(or D) HG-COOH.

XX denotes hyper-variable amino acids at positions 12 and 13, which determine the specificity in base recognition.

In other words, the 12th and 13th amino acids of the TALE-repeat module recognize a single specific nucleic acid. When the XX are HD, the TALE-repeat module recognizes a base Cytosine (C) (SEQ ID NO: 24, 40, 41, 42, 43, or 44). When the XX are NG, the TALE-repeat module recognizes Thymine (T) (SEQ ID NO: 25, 45, 46, 47, 48, or 49). When the XX are NI, the TALE-repeat module recognizes Alanine (A) (SEQ ID NO: 26, 50, 51, 52, 53, or 54). When the XX are NN, the TALE-repeat module recognizes Guanine (SEQ ID NO: 27, 55, 56, 57, 58, or 59).

The amino acids sequence of the present invention is represented by abbreviation of amino acid residues following the IUPAC-IUB nomenclature, as shown below (Table 1).

TABLE 1

| | | | |
|---|---|---|---|
| Alanine | A | Arginine | R |
| Asparagine | N | Aspartic acid | D |
| Cysteine | C | Glutamic acid | E |
| Glutamine | Q | Glycine | G |
| Histidine | H | Isoleucine | I |
| Leucine | L | Lysine | K |
| Methionine | M | Phenylalanine | F |
| Proline | P | Serine | S |
| Threonine | T | Tryptophan | W |
| Tyrosine | Y | Valine | V |

The TALE domains of TALEN comprise one or more tandemly arrayed TALE-repeat modules, each of which recognizes 1 bp (base-pair) sub-site. Unlike zinc finger modules, which recognize 3 by sub-sites, each TALE-repeat module that constitutes TALEs interacts with a single base. Because there are at least four different repeat modules, each preferentially recognizing one of the four bases, it is possible to make designed TALEs (dTALEs) that specifically bind to any predetermined DNA sequence. In other words, only four different modules are needed to make TALENs, whereas up to 64 different zinc finger modules, each corresponding to one of the 64 triplet bases, are required to assemble zinc finger arrays. Although many zinc fingers with exquisite specificities are now used to make ZFNs, the lack of reliable zinc fingers that recognize certain 3-bp subsites, especially CNN and ANN triplets, has been a serious limiting factor. Thus, ZFNs may not be produced that recognize target sites composed of these triplets. Due to this and other limitations such as the context sensitivity of zinc finger-DNA interactions, the target-site density of ZFNs is approximately one per 100 to 1,000 bp, depending on the method of ZFN construction. The gene that has been most densely targeted using

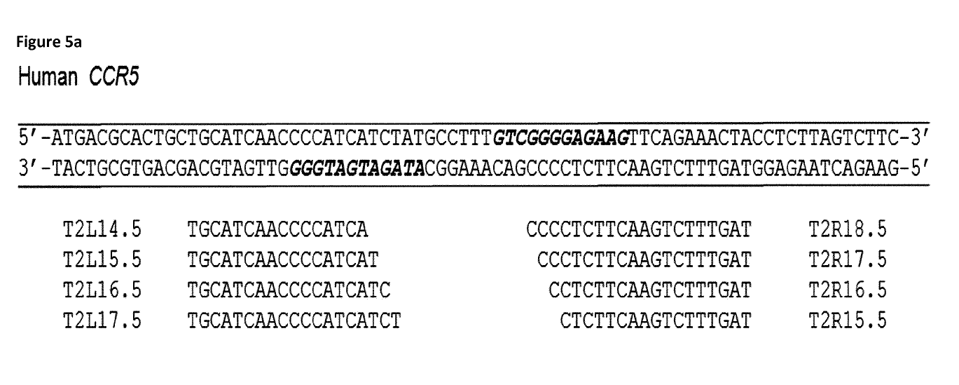

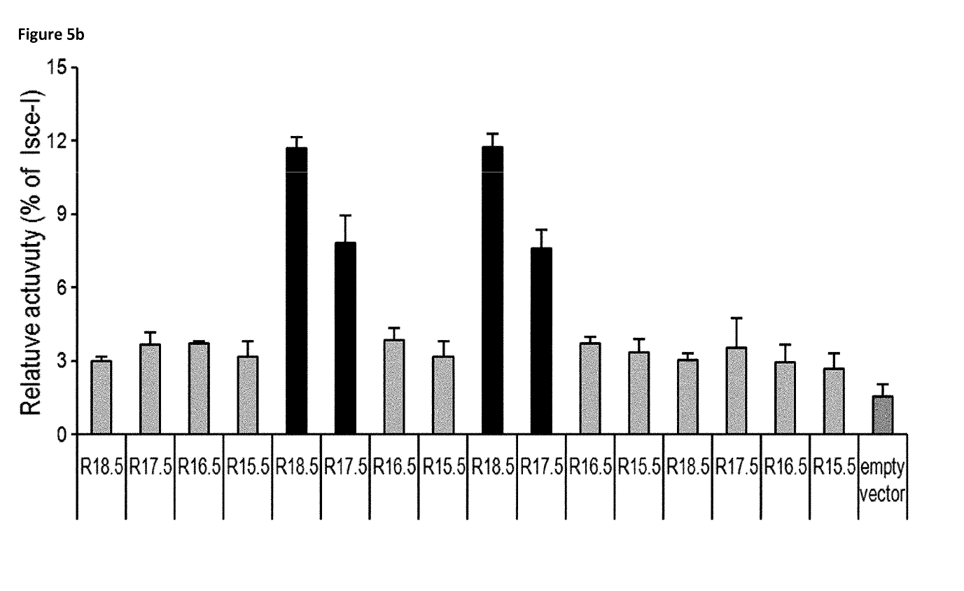

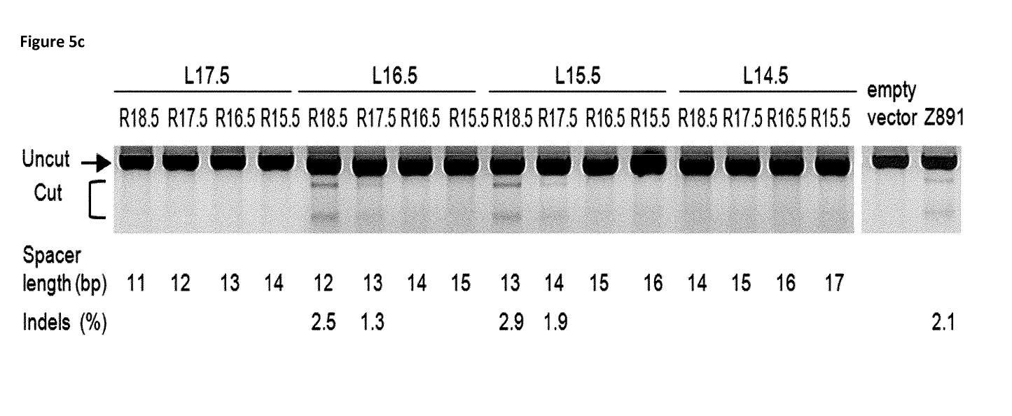

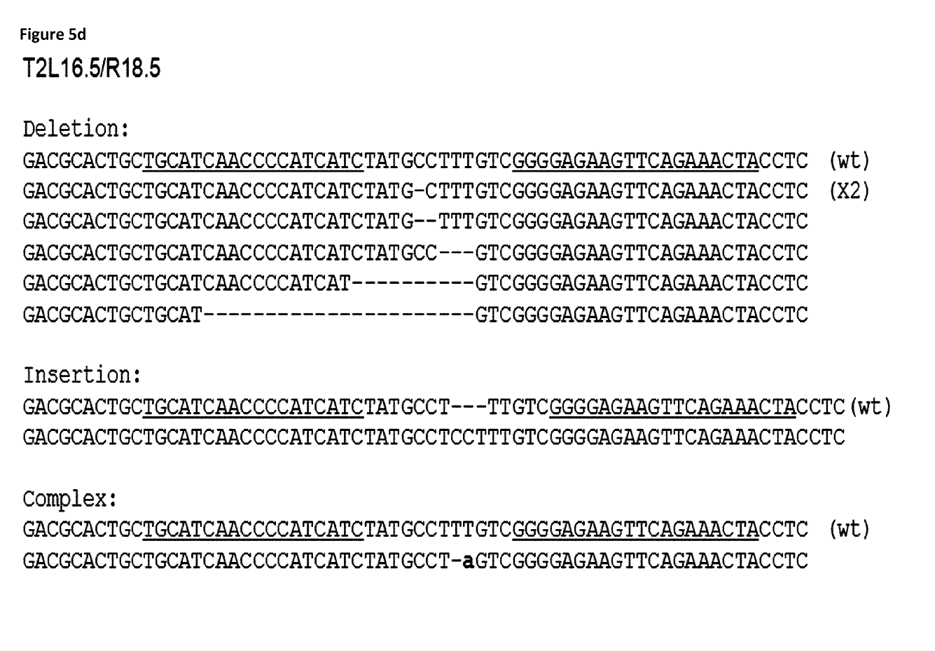

ZFNs reported thus far is human CCR5. In total, 9 functional ZFN pairs (including ZFN-215 and Z891 used in this study) that recognize various sites within the 1 kbp coding region have been produced. This low density is not much of a problem if the aim is to knock out protein-coding genes but does not allow precise manipulation of the genome (such as selective removal of an enhancer element, a promoter, or a miRNA gene) because these targets are too small. TALENs are free of these limitations; TALEN pairs that comprises overlapping arrays of TALE repeats induced mutations at adjacent positions (FIG. 5c). In principle, DSBs can be generated at every base pair using appropriately designed TALENs, which may allow genome engineering at base pair resolution.

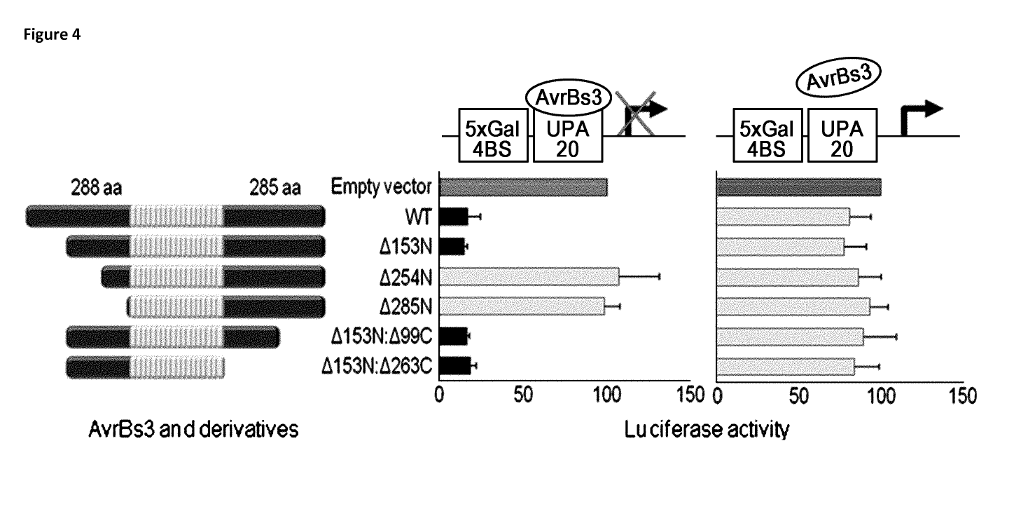

The TALE domain may include the DNA-binding domain of TALEs, and preferably, include at least 135 amino acids sequences of SEQ ID NO: 28, but it is not limited thereto. The 135 amino acids may exist upstream of the TALE-repeat modules. In the specific example, the present inventors found the minimal DNA-binding domain of TALE, which is at least 135 amino acids upstream of the repeat modules (FIG. 4).

As used herein, the term “cleavage” refers to the breakage of the covalent backbone of a nucleotide molecule, and the term “cleavage domain” refers to a polypeptide sequence which possesses catalytic activity for nucleotide cleavage.

The cleavage domain can be obtained from any endo- or exonuclease. Exemplary endonucleases from which a cleavage domain can be derived include, but are not limited to, restriction endonucleases. These enzymes can be used as a source of cleavage domains. In addition, the cleavage domain is able to cleave single-stranded nucleotide sequences, in which double-stranded cleavage can occur depending on the source of cleavage domains. In this regard, the cleavage domain having double-strand cleavage activity may be used as a cleavage half-domain.

Restriction endonucleases are present in many species and are capable of sequence-specific binding to DNA (at a recognition site), and cleaving DNA at or near the site of binding. Certain restriction enzymes (e.g., Type IIs) cleave DNA at sites removed from the recognition site and have separable binding and cleavage domains. For example, the Type IIs enzyme FokI catalyzes double-stranded cleavage of DNA, at 9 nucleotides from its recognition site on one strand and 13 nucleotides from its recognition site on the other.

Examples of the Type IIs restriction enzymes include FokI, AarI, AceIII, AciI, AloI, BaeI, Bbr7I, CdiI, CjePI, EciI, Esp3I, FinI, MboI, sapI, and SspD51, but are not limited thereto, more specifically, see Roberts et al. Nucleic acid Res. 31:418-420 (2003).

As used herein, the term “fusion protein” refers to a polypeptide formed by the joining of two or more different polypeptides through a peptide bond (linker). The polypeptides contain the TALE domain and nucleotide cleavage domain, which can cleave any target site in the nucleotide sequence. Methods for the design and construction of fusion proteins (or polynucleotide encoding fusion protein) may be any methods that are widely known in the art, and the polynucleotide may be inserted into a vector, and the vector may be introduced into a cell. In general, the components of the fusion proteins (e.g., TALE-FokI fusion, TALEN) are arranged such that the TALE domain is nearest the amino terminus (N-terminus) of the fusion protein, and the cleavage half-domain is nearest the carboxy-terminus (C-terminus). This mirrors the relative orientation of the cleavage domain in naturally-occurring dimerizing cleavage domains such as those derived from the FokI enzyme, in which the DNA-binding domain is nearest the amino terminus and the cleavage half-domain is nearest the carboxy terminus.

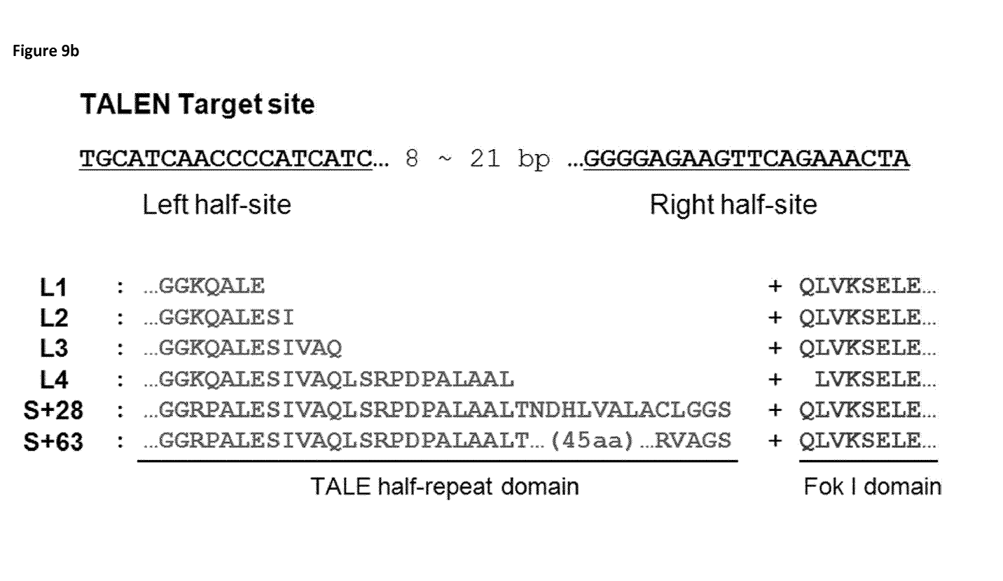

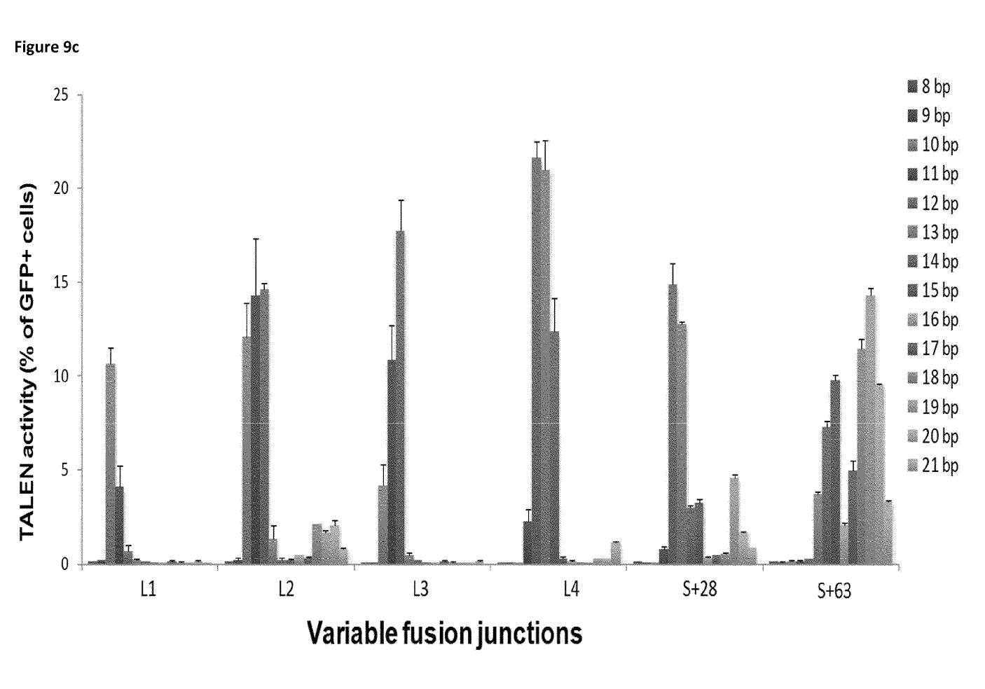

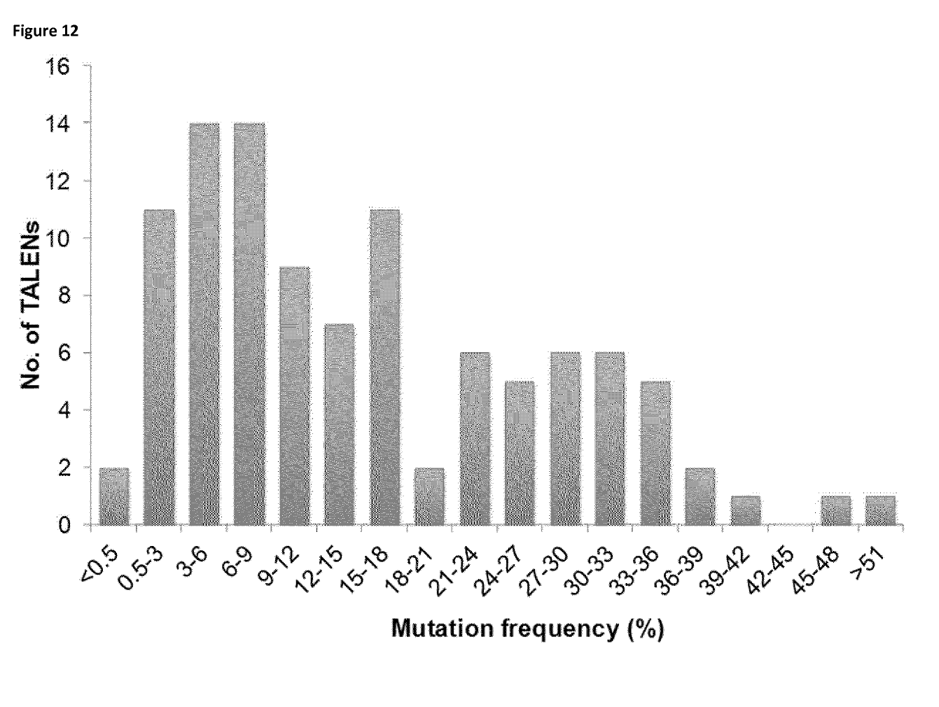

As used herein, the term ‘linker’ refers to a C-terminal of TALE domain. Preferably, the linker may be an amino acid sequence of SEQ ID NO: 60 (L2 linker), 61 (L3 linker), or 62 (L4 linker), or the linker may have no amino acids (L1 linker), but is not limited thereto. TALEN is generally prepared having a basis on TALE domain, and as a result, additional amino acids of TALE domain are left after the TALE-repeat module. The presence of additional amino acids reduces the specificity of TALEN activity. On the other hand, in the present invention, a new TALEN structure has been made having a minimal number of amino acids after the TALE-repeat module and being connected to nucleotide cleavage domain unlike the previous TALEN structure. In one of the Examples, the present inventors found when the linker with a minimal length is used, the specificity and activity of TALEN was improved compared to the previous TALENs represented by S+28 and S+63 (FIGS. 9b and 9c). Particularly, the present inventors have found that a new TALEN architecture induced a mutation in a target gene of the culture human cell with a success rate of over 98% (FIG. 12).

The TALENs comprise the TALE domain and nucleotide cleavage domain, and the TALE domain and the nucleotide cleavage domain are linked by a linker. The length of the linker may be in a range from 0 to 16 amino acids, preferably 2 to 16 amino acids, more preferably 2, 5, 16 amino acids, but it is not limited thereto.

TALEN may function as a dimer, for example homodimers or heterodimers, to introduce DNA double strand breaks, thereby achieving the desired object of the present invention. The dimer may form homodimer of TALEN/TALEN or heterodimer of TALEN/ZFN.

In general, because TALEN functions as a dimer, two TALEN monomers need to be prepared to target a single DNA site. Each of the two monomeric TALENs recognizes one of two half-sites in different DNA strands, which are separated from each other by a 9- or 14-bp spacer. The fusion protein may be designed to have a 9-to 14-bp long spacer between the first half site and second half site, where two TALE domains of the fusion dimer protein bind respectively. Preferably, the spacer may have a length of 10- to 14-bp, more preferably 12- to 14-bp, but is not limited thereto.

If TALEN has the L1 linker, namely has no linker, the TALEN may have a 10-bp long spacer preferably. If TALEN has the L2 linker (SEQ ID NO: 60), the TALEN may have a 10-to 12-bp long spacer. If TALEN has the L3 linker (SEQ ID NO: 61), the TALEN may have a 12 by long spacer. If TALEN has the L4 linker (SEQ ID NO: 62), the TALEN may have a 12-to 14-bp long spacer. In one of the Examples, the present inventors found when the linker is changed, the specific spacer of TALEN was changed according to the linker (FIGS. 9b and 9c).

In accordance with another aspect, the present invention relates to a nucleotide encoding the fusion proteins.

In accordance with another aspect, the present invention relates to a recombination kit for cleavage, replacement or modification of DNA sequences in a targeted region, comprising one or more pairs of the fusion proteins.

In general, because TALENs function as dimers, two TALEN monomers or ZFN and TALEN monomers need to be prepared to target a single DNA site. For a single half-site, multiple monomeric TALENs can be designed, which comprise different sets of TALE-repeat modules with identical or similar DNA-binding specificities. The single site can be targeted with many combinatorial TALEN pairs or ZFN/TALEN pairs.

As used herein, the term “replacement” can be understood to represent replacement of one nucleotide sequence by another, (i.e., replacement of a sequence in the informational sense), and does not necessarily require physical or chemical replacement of one polynucleotide by another. As used herein, the term “modification” means a change in the DNA sequence by mutation or nonhomologous end joining. The mutations include point mutations, substitutions, deletions, insertions or the like. The replacement or modification can replace or change a nucleotide having incomplete genetic information with a nucleotide having complete genetic information. The peptide encoded by the nucleotide sequence can also be functionally inactivated by the mutation. By this means, the TAL effector nuclease can be used as a tool for gene therapy.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

In accordance with another aspect, the present invention relates to a cell comprising the fusion proteins.

The cell maybe prokaryotic cells such as E. coli, or eukaryotic cells such as yeast, fungus, protozoa, higher plant, and insect, or amphibian cells, or mammalian cells such as CHO, HeLa, HEK293, and COS-1, for example, cultured cells (in vitro), graft cells and primary cell culture (in vitro and ex vivo), and in vivo cells, and also mammalian cells including human, which are commonly used in the art, without limitation.

In accordance with another aspect, the present invention relates to a method for deletion, duplication, inversion, replacement, insertion or rearrangement of genomic DNA, comprising the step of cleaving specific sites in a genome using the fusion proteins.

The one pair of TAL effector nuclease may be separated by 9- to 14-bp spacers, and the spacers is the length between the half-sites bound TALE domain.

### EXAMPLES

Hereinafter, the present invention will be described in more detail with reference to Examples. However, these Examples are for illustrative purposes only, and the invention is not intended to be limited by these Examples.

### Methods

### Example 1

### Construction of Truncated Forms of AvrBs3

The AvrBs3 gene was amplified from Xhanthomonas cempestris pv. Vesicatoria (Xcv) (RDA Genebank, Korea, KACC no. 11157) using Phusion DNA polymerase (Finnzymes, Finland) and primer sets AB-F and AB-R (Table 2). The PCR product was digested with EcoRl/Xhol and subcloned into p3, a derivative of pCDNA3 (Invitrogen). DNA segments encoding truncated forms of AvrBs3 were amplified using appropriate primer sets: A153N (AB-N153F and AB-R), A254N (AB-N254F and AB-R), A285N (AB-N285F and AB-R), A153N:A99C (AB-N153F and AB-C99R), and A153N:A258C (AB-N153F and AB-C263R). Each PCR product was digested with EcoRl/Xhol and subcloned into p3. All the primers used in this study are listed in Table 2.

TABLE 2

| | | SEQ ID |
|---|---|---|
| Label | Sequence | NO. |
| AB-F | 5′-TTCGAATTCAAATGGATCCCATTCGTTCGCG-3′ | 11 |
| AB-R | 5′-TTGCTCGAGTCACTGAGGCAATAGCTCCATC-3′ | 12 |
| AB-N153F | 5′-TTCGAATTCAAGATCTACGCACG-3′ | 13 |
| AB-N254F | 5′-TTCGAATTCAATTGGACACAGGC-3′ | 14 |
| AB-N285F | 5′-TTCGAATTCAACCCCTGAACCTG-3′ | 15 |
| AB-C99R | 5′-TTACTCGAGTCAGCTGCTTGCCC-3′ | 16 |
| AB-C263R | 5′-TTGCTCGAGCAACGCGGCCAACGC-3′ | 17 |
| UPA20F | 5′-AATTCATCTTTATATAAACCTGACCCTTTGTGACGAGCT-3′ | 18 |
| UPA20R | 5′-CGTCACAAAGGGTCAGGTTTATATAAAGATG-3′ | 19 |

### Example 2

### Transcriptional Repression Assay

The luciferase reporter plasmid, pGL3-UPA20/Inr, was constructed by replacing the adenovirus major late TATA box in pGL3-TATA/Inr (Kim at al, Transcriptional repression by zinc finger peptides. Exploring the potential for applications in gene therapy. J Biol Chem 272, 29795-29800 (1997)) with the UPA20 box using oligonucleotide pairs (UPA2OF and UPA2OR, Table 2). The transcriptional repression assay was performed as described (Kim at al, Transcriptional repression by zinc finger peptides. Exploring the potential for applications in gene therapy. J Biol Chem 272, 29795-29800 (1997)). Briefly, HEK293T/17 cells (2×10⁵) pre-cultured in a 24 well plate were co-transfected with the following plasmids: empty vector, p3, or each of the expression plasmids encoding AvrBs3 derivatives (400 ng), the reporter plasmid [pGL3-UPA20/Inr or pGL3-TATA/Inr (100 ng)], activator-encoding plasmid [Ga14-VP16 (100 ng)], and carrier plasmid [pUC19 (200 ng)]. After 48 h of incubation, cells were lysed in 1× lysis buffer (50 μl) (Promega), and the luciferase activity in the cell lysate (2 μl) was measured using the luciferase assay reagent (25 μl) (Promega).

### Example 3

### TALEN Expression Plasmids

Oligonucleotides that encode each TALE repeat module were synthesized and subcloned into the Xbal/Nhel site in p3. The DNA sequence of a module termed HD is as follows:

(SEQ ID NO: 20)
5′-tctagagaccgtgcagcgcctgctgcccgtgctgtgccaggcccacggcctgacccccgag
caggtggtggccatcgccagccacgacggcggcaagcaggcgctagc-3′.

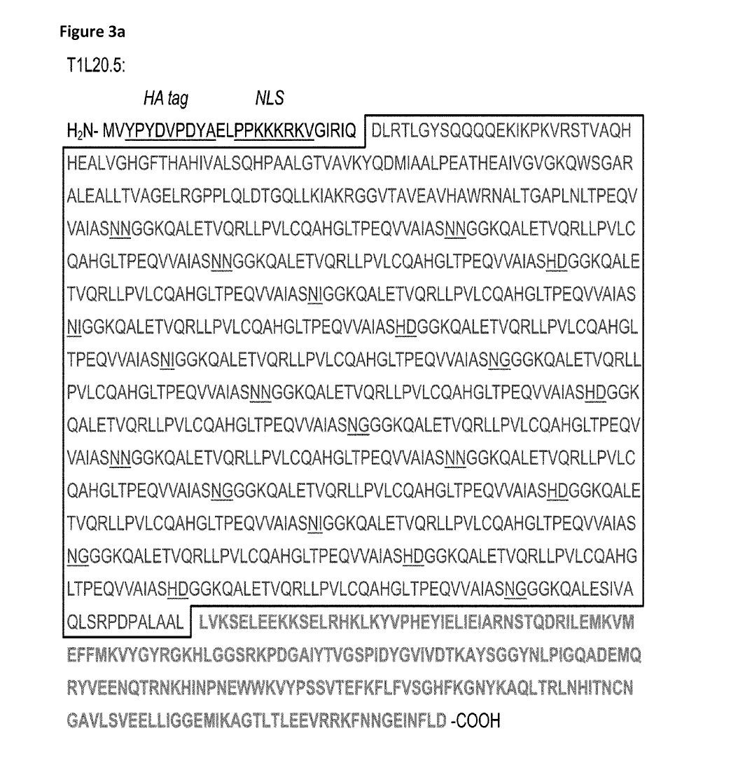

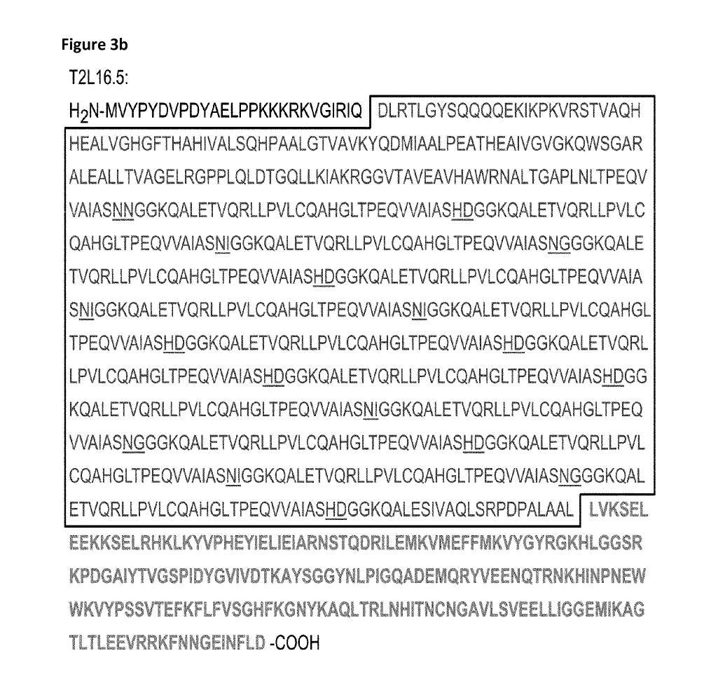

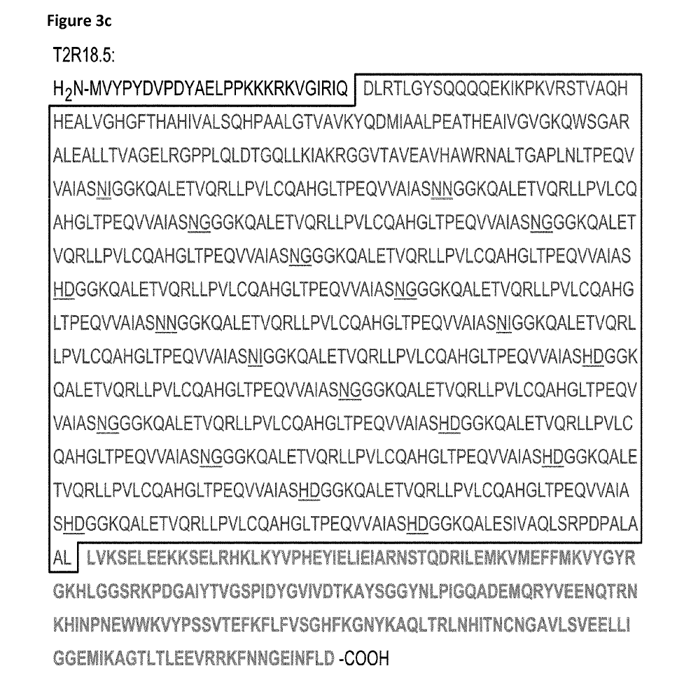

Underlined sequences were changed to “aatggc”, “aatatt”, or “aataac” to encode NG, NI, or NN, respectively (SEQ ID NOs: 21, 22 and 23). One plasmid was digested with XbaI and XhoI to yield a vector backbone and the other with NheI and XhoI to yield an insert segment. To create a plasmid encoding a two-repeat array, the insert segment was ligated with the vector backbone. The resulting plasmids were subjected to the next round of subcloning using the same sets of restriction enzymes. Finally, modularly-assembled repeat arrays were subcloned into an expression vector that encodes the A153 N-terminal domain of AvrBs3 at the N terminus and the Fokl nuclease domain at the C terminus (FIG. 2) to create TALEN expression vectors. The complete amino acid sequences of CCR5-targeting TALENs are shown in FIG. 3.

### Example 4

### Cell-Based Luciferase Assay Using the Single-Strand Annealing System

HEK293T/17 (ATCC, CRL-11268TM) cells were maintained in Dulbecco's modified Eagle medium (Welgene Biotech.) supplemented with 100 units/ml penicillin, 100 μg/ml streptomycin, and 10% fetal bovine serum (Welgene Biotech.). Each pair of TALEN or ZFN expression plasmids (400 ng each) was transfected into 2×10⁵ reporter cells/well in a 24-well plate format using Lipofectamine 2000 (Invitrogen). After 48 h, the luciferase gene was induced by incubation with doxycycline (1 μg/ml). After 24 h of incubation, cells were lysed in 1× lysis buffer (50 μl) (Promega), and the luciferase activity in the cell lysate (2 μl) was determined using the luciferase assay reagent (25 μl) (Promega).

### Example 5

### T7E1 Assay

HEK293T/17 cells (2×10⁵) pre-cultured in a 24 well plate were transfected with two plasmids encoding a TALEN or ZFN pair (400 ng each) using Lipofectamine 2000 (Invitrogen). After 72 h of incubation, genomic DNA was extracted from the transfected cells using the G-spin™ Genomic DNA Extraction Kit (iNtRON BIOTECHNOLOGY). Purified genomic DNA samples were subjected to the T7 endonuclease I (T7E1) assay as described previously (Kim et al., Targeted genome editing in human cells with zinc finger nucleases constructed via modular assembly. Genome Res 19, 1279-1288 (2009)).

### Example 6

### PCR Analysis for Genomic Deletion and Sequencing of the Breakpoint Junctions

Genomic DNA (50 ng per reaction) was subjected to PCR analysis using Taq DNA polymerase (GeneAll Biotech) and appropriate primers as described previously (Lee et al. Targeted chromosomal deletions in human cells using zinc finger nucleases. Genome Res 20, 81-89 (2010)). For sequencing analysis, PCR products corresponding to genomic deletions were purified using the QIAquick Gel Extraction Kit (QIAGEN) and cloned into the T-Blunt vector using the T-Blunt PCR Cloning Kit (SolGent). Cloned plasmids were sequenced using M13 primers or primers used for PCR amplification.

### Example 7

### Construction of Plasmids for Expressing Golden-Gate Assembly of TALENs

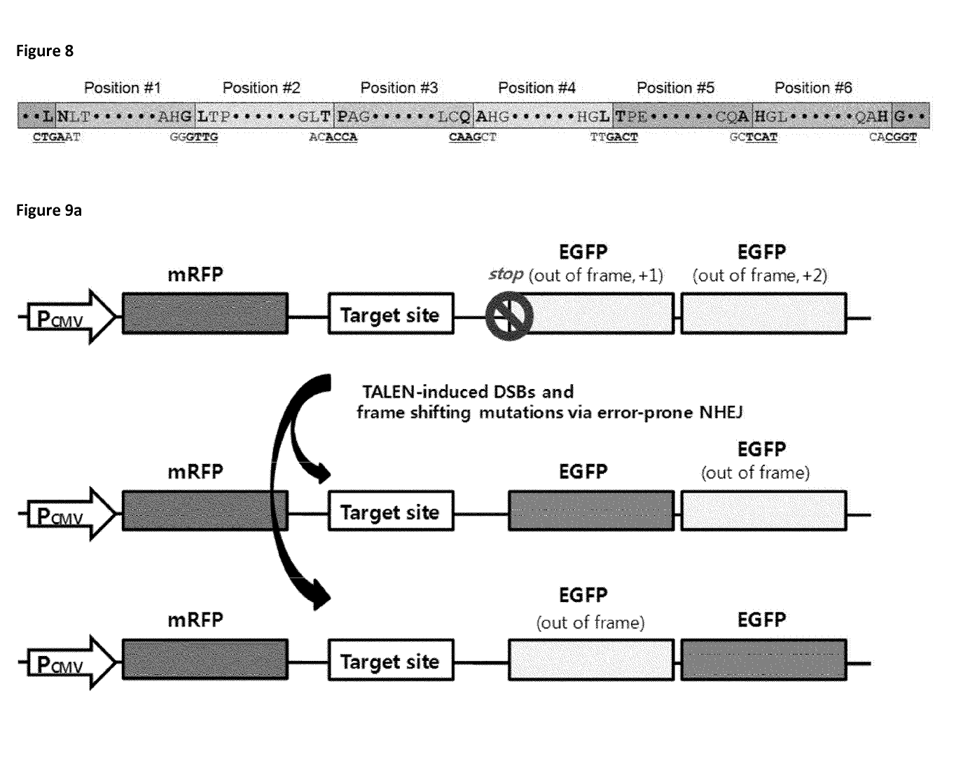

The 424 TALE array plasmids were constructed using a total of 84 TALE plasmids which include 64 tripartite, 16 bipartite, and 4 monopartite arrays having a combinations of NN, HD, NI, and NG RVD modules that were synthesized by GenScript Corporation. To avoid undesired results, RVD modules that target rare human codons were excluded and the maximum sequence identity among different RVDs is limited to 81%. Each of the 84 plasmids was amplified by PCR with a carefully selected primer set that confers different overhang upon restriction digestion with BsaI at each of the six TALE array positions. The PCR amplicons were then subcloned into a vector with the kanamycin-resistance selection marker. The 8 FokI expression plasmids consist of an ampicillin-resistance gene, a CMV promoter, a HA epitope tag, a nuclear localization signal, N-terminal 135 amino acids of AvrBs3, one of the four RVD half-repeats, and the Sharkey FokI domain (DAS or RR) (Guo, J., et al., 3rd Directed evolution of an enhanced and highly efficient FokI cleavage domain for zinc finger nucleases. J Mol Biol 400, 96-107 (2010)). The amino acid and DNA sequences of a TALEN pair that was assembled using the above system are shown in FIG. 8 as SEQ ID NO: 38 to 39.

In more detail, all steps in making TALEN assembly were performed in 96-well plates. In each plate, 47 pairs of TALENs were assembled and one pair of FokI vector alone was included as a negative control. Overall, the present one-step Golden-Gate system involves 424 TALE array plasmids (6×64 tripartite arrays, 2×16 bipartite arrays, and 2×4 monopartite arrays). Each TALE array was numbered as shown in Table 3. These numbers were used to choose the appropriate arrays for assembling TALEN plasmids.

TABLE 3

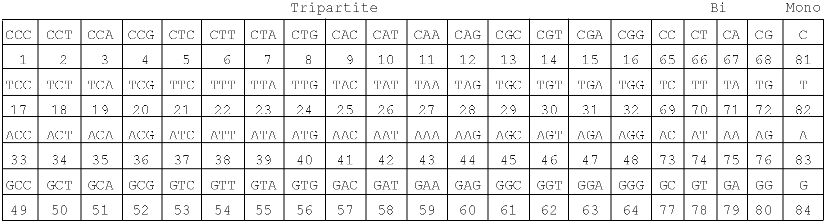

For example, the sequence of left half-site, “5′-TGGGGGAGGTGGCGAGGAAC”, can be divided into 8 parts (the first T, GGG, GGA, GGT, GGC, GAC, GAA, and the last C). The first T and last C are not recognized by TALE arrays. To assemble a TALEN subunit targeting the above sequence, the following arrays are chosen to be inserted into an expression vector: position1-#64+position2-#63+position3-#62+position4-#61+position5-#57+position6-#5930 the FokI expression vector that contains C-specific half-repeat. A detailed protocol is described below:

1) Six TALE array plasmids and a FokI expression vector are mixed in each well as follows for preparing a 20 μl restriction-ligation reaction:

1.0 μl TALE array vectors (50 ng/μl each)

0.5 μl FokI expressing vector (50 ng/μl)

0.5 μl BsaI (New England BioLabs, 10 U/μl)

2.0 μl 10×T4 DNA Ligase Reaction Buffer

0.1 μl T4 DNA Ligase (New England BioLabs, 2000 U/μl)

10.9 μl ddH₂O 2) The restriction-ligation reaction is carried using a thermocycler with the following condition:

20 cycles for 37° C. 5 min and 16° C. 5 min

50° C. 15 min

80° C. 5 min

3) After the thermocycling reaction, the reaction mixture (6 μl) from each well is transformed into the chemically competent DH5a cells (30 μl). Subsequently, the transformed cells are inoculated with LBmedium (800 μl) containing ampicillin (50 μg/ml) in Flat-Bottom Blocks (Qiagen). The transformants in 96-well blocks are incubated overnight at 37° C. with vigorous shaking.

4) Two sets of glycerol stock of E. coli are prepared by mixing the E. coli culture in LB (50 μl) with 60% glycerol (150 μl); each stock is stored at −80° C.

### Example 8

### Culturing and Transfection of Mammalian Cell

HEK 293T/17 (ATCC, CRL-11268) and HeLa cells (ATCC, CCL-2TM) were stored in Dulbecco's modified Eagle's medium (DMEM) supplemented with 100 units/mL penicillin, 100 μg/mL streptomycin, 0.1 mM nonessential amino acids, and 10% fetal bovine serum (FBS). About 400,000 HEK 293 cells were transfected with 3 μl of polyethylenimine and 1 μg of plasmid DNA in each of the 24-well plate. About 200,000 HeLa cells were transfected with Lipofectamine 2000 (Invitrogen) following the manufacturer's protocol.

### Example 9

### Measurement of Genome-Editing Activity of TALENs Using T7E1 Assay

After 3 days of transfection, genomic DNA was extracted by using G-DEX IIc Genomic DNA Extraction Kit (iNtRON). TALEN target sites were PCR-amplified. For sequencing analysis, PCR products were purified and subcloned into a T-Blunt vector (SolGent) and subjected to dideoxy DNA sequencing. The 17E1 analysis was performed as described in Kim, H. J., et al., (Targeted genome editing in human cells with zinc finger nucleases constructed via modular assembly. Genome Res 19, 1279-1288 (2009)).

### EXAMPLE 10

### TALEN-Induced Genome Rearrangements

Genomic DNA was isolated from the cells transfected with two pairs of TALENs. To determine the frequency of chromosomal rearrangements, genomic DNA was diluted in a serial dilution, which was then subjected to a digital PCR using selected primer set. The results were analyzed using the Extreme Limiting Dilution Analysis program as described in Lee, H. J., et al., (Targeted chromosomal deletions in human cells using zinc finger nucleases. Genome Res 20, 81-89 (2010)). The breakpoint junctions were analyzed by a dideoxy DNA sequencing.

Results

### Experimental Example 1

### Determination of the Minimal DNA-Binding Domain of TALE

The minimal DNA-binding domain of a prototype TALE protein, AvrBs3 was determined, by preparing a series of truncated forms from either the N- or C-terminus (FIG. 4). The DNA-binding activity of these truncated TALE proteins was assessed in HEK293 cells using a transcriptional repression assay. In this assay, plasmids that encode truncated or full-length TALEs are co-transfected with a reporter plasmid that encodes the firefly luciferase gene. Because the AvrBs3 target site, termed UPA20, is incorporated near the transcriptional start site, proteins able to bind to this site could inhibit the transcription of the reporter gene. It was found that the C-terminal segment downstream of the TALE repeat domains could be deleted without affecting the DNA-binding activity of AvrBs3. In contrast, at least 135 amino acids upstream of the repeat domains must be retained for truncated TALEs to bind to the target site.

### Experimental Example 2

### Preparation of TALEN

TALENs were then constructed by fusing custom-designed minimal dTALE-repeat domains to the N-terminus of the FokI nuclease domain. These TALE-repeat domains were designed to recognize 11- to 18-bp DNA sequences at the coding region of the human chemokine receptor 5 (CCR5) gene, which encodes a co-receptor for HIV. Because an optimal linker was unknown, a series of TALE-FokI fusions with different junctions was prepared by linking each dTALE to various amino acid residues in the appropriate region of the FokI nuclease domain (FIG. 1c). Instead of testing TALEN/TALEN dimers directly, TALEN/ZFN pairs were first tested (because the FokI domain must be dimerized to cleave DNA, we expect that TALENs, like ZFNs, function as dimers.). To this end, ZFN-215, a ZFN pair that induces targeted mutations at the CCR5 gene was chosen (Perez, E.E. et al. Establishment of HIV-1 resistance in CD4+ T cells by genome editing using zinc-finger nucleases. Nat Biotechnol 26, 808-816 (2008)), and one of the ZFN monomers (termed 215L) was replaced with a series of TALEN constructs. Thus a TALEN/ZFN pair consists of one of the TALEN constructs and the other subunit of ZFN-215 (termed 215R). Whether these TALEN/ZFN pairs could induce a DSB using a cell-based reporter assay in which the functional luciferase gene is restored via single-strand annealing after DNA cleavage was then tested. Among the 56 combinatorial pairs (=8 spacers×7 linkers) tested, only one TALEN/ZFN pair resulted in significant luciferase activity compared to the negative controls such as an empty vector or 215R alone (p<0.01, Student's t-test) (FIG. 1d). The active TALEN identified in this assay (termed T1L11.5) consists of 11.5 TALE repeats (the last repeat domain is considered to be a half-repeat domain because it has a limited homology with other repeats) and recognizes a 13-bp half-site (including the invariant T at position 0), which is separated from the 215R half-site by a spacer of 9 by in length. To enhance the activity of the TALEN/ZFN pair, more repeats at the N terminus were added to make an elongated TALEN termed T1L20.5 that consists of 20.5 repeats and recognizes a 22-bp DNA sequence. This TALEN paired with 215R showed significantly higher activity (p<0.05) compared to the original TALEN/ZFN pair in the reporter assay (FIG. 1d).

### Experimental Example 3

### Analysis of Inducing Small Insertions and Deletions by TALEN/ZFN Pairs

Next, it was investigated whether these active TALEN/ZFN pairs could, indeed, induce small insertions and deletions (indels) at the endogenous CCR5 site, characteristic of error-prone DSB repair via NHEJ, using mismatch-sensitive T7 endonuclease 114 (T7E1) (FIG. 1e). PCR amplicons from cells transfected with plasmids encoding the TALEN/ZFN pairs were partially cleaved at the expected position, indicating the presence of indels at the CCR5 site. In line with the results obtained using the cell-based luciferase assay, the elongated TALEN, L20.5, was more active than L11.5. DNA sequencing analysis confirmed the induction of indels at the spacer region (FIG. 1f). These results demonstrate that TALENs can replace ZFNs and that TALEN/ZFN pairs induce bona-fide genome modifications in cultured human cells.

### Experimental Example 4

### Analysis of Inducing Targeted Mutagenesis in Human Cells by TALEN/TALEN Pairs

It was then investigated whether TALEN/TALEN pairs can also induce targeted mutagenesis in human cells. First, an educated guess was made of the spacer length that would allow DNA cleavage. It was reasoned that, because the active TALEN/ZFN pairs bind to two half-sites separated by a 9-bp spacer, whereas typical ZFN pairs recognize two half-sites separated by a 5- or 6-bp spacer, the TALEN subunit in the TALEN/ZFN pairs must have required 3 to 4 additional bases in the spacer. This suggests that the optimal binding sites for TALEN/TALEN dimers may have a 11- to 14-bp spacer.

To test this idea, another site was focused on at the CCR5 locus, which had also been successfully targeted by a ZFN pair, termed Z891, in a previous study (Kim, H. J. et al., Targeted genome editing in human cells with zinc finger nucleases constructed via modular assembly. Genome Res 19, 1279-1288 (2009)), and a series of TALENs that were designed to recognize overlapping DNA sequences were synthesized (FIG. 5a). All of these TALENs contain the same linker as the two TALENs that successfully replaced 215L. Each of the left-side TALEN monomers was paired with each of the right-side monomers, and the activity of each pair was measured using the cell-based luciferase assay. Among the 16 combinatorial TALEN pairs tested, only four pairs resulted in significant luciferase activities compared to the negative control (FIG. 5b). These four pairs bind to half-sites separated by 12- to 14-bp spacers, in good agreement with our educated guess.

### Experimental Example 5

### Analysis of Inducing Genome Modifications at the Endogenous Site by TALEN Pairs

The T7E1 assay were then used to investigate whether these TALEN pairs could induce genome modifications at the endogenous site. Only the four active TALEN pairs identified using the luciferase assay showed T7E1-driven DNA cleavage, indicating the induction of indels at the CCR5 site (FIG. 5c). Based on the fractions of DNA cleavage, the mutation frequencies of TALEN pairs at the endogenous site were estimated to be in the range of 1 to 3%, which is on par with that of Z891 (20), the ZFN pair that targets the same site. To confirm targeted genomic mutagenesis by the L16.5/R18.5 TALEN pair, the DNA sequences of PCR products representing the appropriate genomic region were determined and it was found that indels were induced in and around the spacer region (FIG. 5d), reminiscent of mutagenic patterns induced by ZFNs, at a frequency of 9% (8 indels/92 clones). In contrast, each TALEN monomer alone failed to show any genome-editing activity (assay sensitivity, ˜1%).

### Experimental Example 6

### Analysis of Inducing Large Chromosomal Deletions by TALEN/ZFN or TALEN Pairs

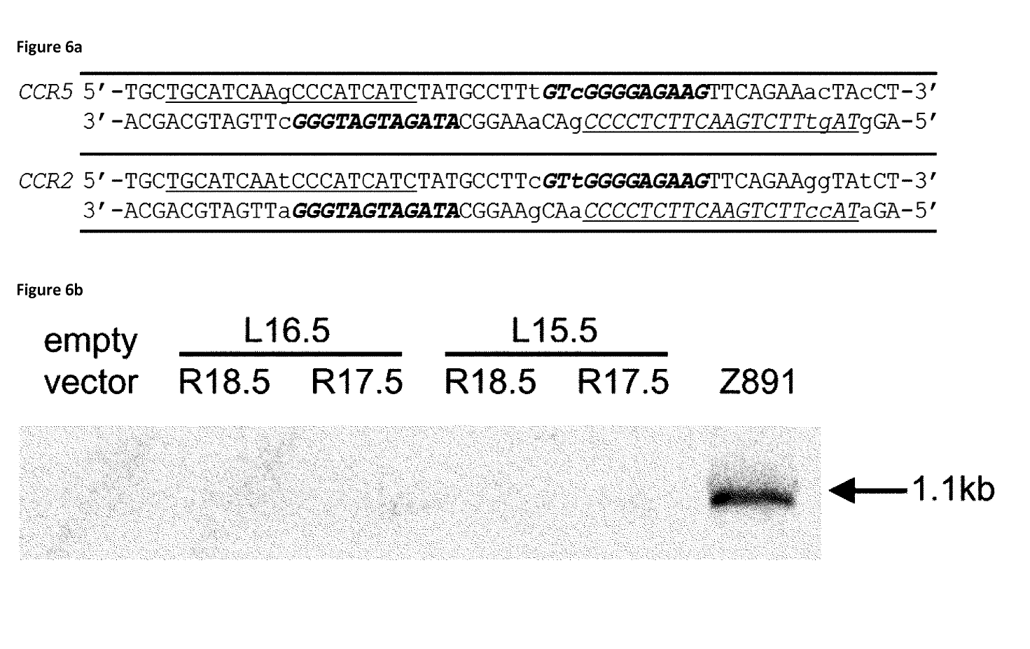

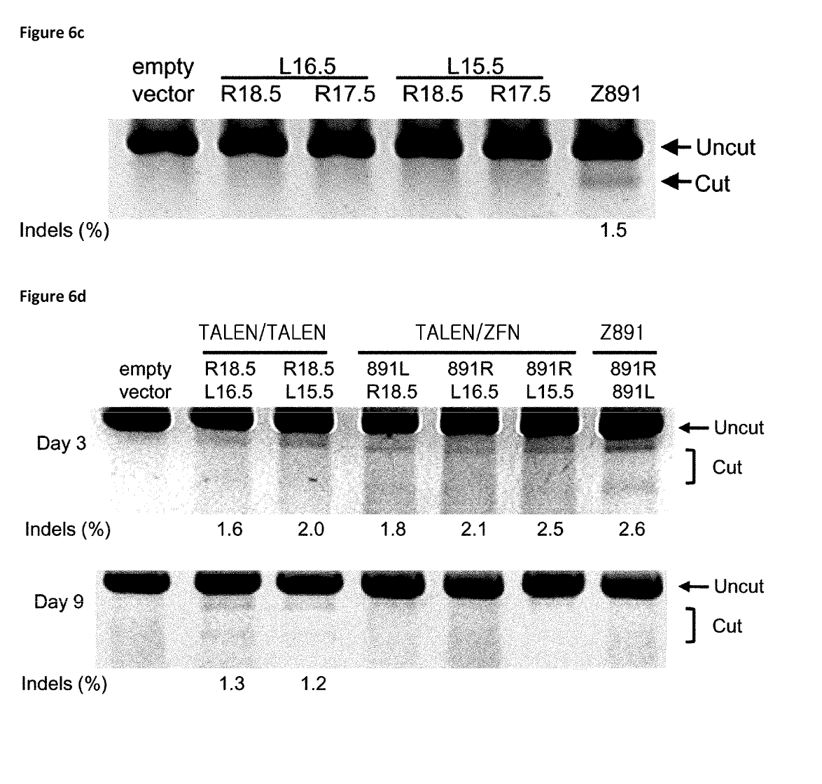

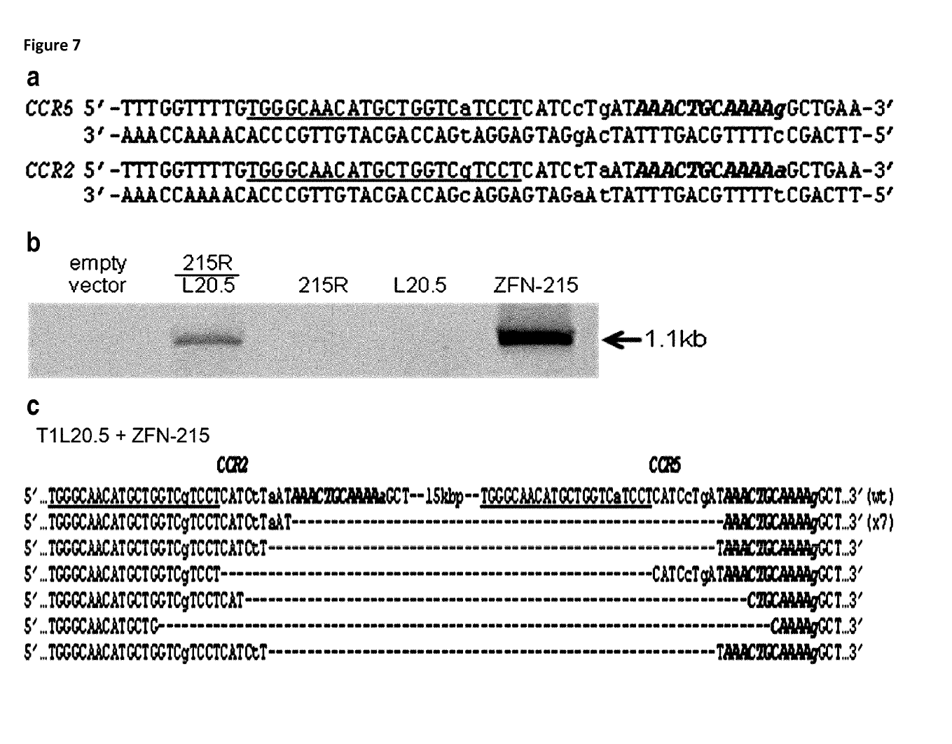

Whether TALEN/ZFN or TALEN pairs can induce large chromosomal deletions as observed previously with ZFN pairs was also tested (Lee, H. J. et al., Targeted chromosomal deletions in human cells using zinc finger nucleases. Genome Res 20, 81-89 (2010). Both ZFN-215 and Z891 used in this study recognize two highly homologous sites, one at the CCR5 locus and the other at the CCR2 locus (FIG. 6a), and efficiently induce targeted deletions of the intervening 15-kbp DNA segments between the two sites. PCR were used to detect the presence of deletion junctions in the cells transfected with plasmids encoding TALEN/ZFN or TALEN pairs. Only the T1L20.5/215R hybrid pair targeting the ZFN-215 site but not the TALEN pairs targeting the Z891 site induced 15-kbp deletions (detection limit<0.01%) (FIGS. 6b and 7). PCR products were cloned and sequenced, which confirmed specific deletions of 15-kbp DNA segments between the CCR2 and CCR5 sites using the TALEN/ZFN pair (FIG. 7). This result shows that the TALEN/ZFN hybrid pair can induce two concurrent DSBs, which give rise to large chromosomal deletions and that the TALEN monomer, T1L20.5, can tolerate a single-base mismatch at the CCR2 site, which raises the possibility that TALENs, like ZFNs, may elicit off-target mutations at unintended sites.

### Experimental Example 7

### Analysis of Off-Target Effects of TALEN Pairs

To investigate off-target effects of TALEN pairs, potential off-target sites were first searched for, in the human genome, whose sequences are similar to that of the CCR5 site (Table 4). Table 4 shows potential off-target sites of the CCR5-targeting TALEN pair in the human genome. Bioinformatic analysis was performed to search for sites that are most similar to the CCR5 target site. All potential half-sites for the two TALEN monomers, T2L16.5 and T2R18.5, were identified in the human genome, allowing up to 5-base mismatches from the CCR5 target site. Because TALENs can function as either homodimers or heterodimers, these two possibilities were considered. Two-half sites separated by a 12- to 14-bp spacer were identified and ranked based on the similarity score, which was calculated as the product of the percent identify at the two half-sites. Mismatching bases are shown in lowercase letters. The top 10 potential off-target sites are listed.

| | | | | | | | | | Homodimer |
|---|---|---|---|---|---|---|---|---|---|
| | | Chromo- | | Left half-site | Mis- | Right half-site | Mis- | Spacer | or |
| Rank | Score | some | Gene | (5′ to 3′) | match | (5′ to 3′) | match | (bp) | Heterodimer |
| Intended | 1 | 3 | CCR5 | TGCATCAACCCCATCATC | 0 | TAGTTTCTGAACTTCTCCCC | 0 | 12 | Heterodimer |
| 1 | 0.85 | 3 | CCR2 | TGCATCAAtCCCATCATC | 1 | TAccTTCTGAACTTCTCCCC | 2 | 12 | Heterodimer |
| 2 | 0.65 | 3 | CXCR1 | TGCcTgAAtCCtcTCATC | 5 | TAtcTTCTGAACTTCTCCCC | 2 | 12 | Heterodimer |
| 3 | 0.63 | 3 | CCR4 | TGCcTtAAtCCCATCATC | 3 | TAcTTgCgaAAtTTCTCCCC | 5 | 12 | Heterodimer |
| 3 | 0.63 | 7 | GPER1 | TGCcTaAACCCCcTCATC | 3 | TtGTccCTGAAggTCTCCCC | 5 | 12 | Heterodimer |
| 5 | 0.58 | 3 | CCR3 | TGCATgAACCCggTgATC | 4 | TAcTTcCgGAACcTCTCtCC | 5 | 12 | Heterodimer |
| 6 | 0.56 | 1 | N/A | TtCtTtAACCCCATtAgC | 5 | aaCATCAACCCCtcCATC | 4 | 12 | Homodimer |
| 6 | 0.56 | 4 | N/A | TGgAgCAAtgCCATtATC | 5 | TGCATCcAaCCttTCATC | 4 | 14 | Homodimer |
| 8 | 0.54 | 3 | CCR1 | TGtgTCAACCCagTgATC | 5 | TAcTTcCgGAACcTCTCaCC | 5 | 12 | Heterodimer |
| 8 | 0.54 | 9 | TLE4 | TtCAgtAtCCCCATCAgC | 5 | gAGTTTCTGtgCTTCTCagC | 5 | 13 | Heterodimer |
| 10 | 0.52 | 6 | BRPF3 | TtCATtAAtCCCcTCATa | 5 | aGCcTCAACttCcTCATC | 5 | 12 | Homodimer |

Because all the ZFNs and TALENs used in this study contain the wild-type FokI domain but not an obligatory heterodimeric FokI domain, sites for binding both homodimeric and heterodimeric enzymes were considered in this analysis. The most similar sequence to the site targeted by the four functional TALEN pairs was found at the CCR2 locus, as expected. The CCR2 off-target site consists of two half-sites, each of which carries one- and two-base mismatches, respectively, with the corresponding half-sites of the CCR5 on-target site (FIG. 6a). The T7E1 assay was used to test whether the TALEN pairs could induce indels at the CCR2 off-target site (FIG. 6c). No mutations were detected at this off-target site, which is in line with the result that these TALEN pairs failed to induce chromosomal deletions as described above. In contrast, Z891, whose recognition sequence at the CCR2 site carries only a single base mismatch, induced both local off-target mutations at the CCR2 site and chromosomal deletions (FIGS. 6b and 6c). Other potential off-target sites were also tested using T7E1 and it was found that the TALEN pairs did not induce any mutations at these sites.

### Experimental Example 8

### Analysis of Cellular Toxicity

One of the most critical limitations of ZFNs is cellular toxicity, which may arise from off-target mutations. Thus, cells that carry ZFN-induced mutations often are growth-impaired and outgrown by unmodified cells, which hampers the isolation of target-modified cells. Because TALENs recognize longer DNA sequences than do typical ZFNs, TALEN pairs may be more specific and have reduced off-target effects and cytotoxicity compared to ZFNs. To test this hypothesis, the T7E1 assay was used to compare the stability of indels induced by TALEN, TALEN/ZFN, and ZFN pairs with one another. It was found that the cleaved DNA bands corresponding to indels disappeared at day 9 after transfection when cells expressed Z891 or ZFN/TALEN hybrid pairs (FIG. 6d). In sharp contrast, these DNA bands persisted at day 9 when cells expressed TALEN pairs. These results indicate that the instability of nuclease-driven indels or cytotoxicity is caused mainly by the ZFN monomers (891R and 891L), and not by the TALEN monomers.

### Experimental Example 9

### Designing Prototype TALENs

The present inventors first optimized the architecture of TALENs by investigating the cleavage activity of TALENs with various fusion junctions where a TALE array is linked to the FokI nuclease domain on the target sites with different spacer lengths. TALENs that work as a dimer recognize two half-sites separated by a spacer and then cleave at the spacer. RFP-GFP reporters, which contain potential target site having a spacer between the RFP- and GFP-encoding DNA sequences, were used to measure the cleavage activity of TALENs in human embryonic kidney (HEK) 293 cells. The GFP sequence is fused with the RFP sequence out of frame. Thus a functional GFP can be expressed only when TALEN induces DSBs at the target site and then repairing of the DSBs by error-prone NHEJ gives rise to indels that often result in frameshift mutations (FIG. 9a). Among the TALENs that were investigated by this assay, ones having 12- to 14-bp long spacer (L4) showed a high cleavage activity at the target site, while ones with less than 12-bp or more than 14-bp long spacer showed no or negligible cleavage activity at the target sites (FIGS. 9b and 9c). In comparison to the two original TALEN constructs that contain longer spacer between the TALE array and the FokI sequence (S+28 and S+63 in FIGS. 9b and 9c) (Miller, J. C. et al. A TALE nuclease architecture for efficient genome editing. NatBiotechnol 29, 143-148 (2011).), the TALEN constructs of the present invention demonstrated a higher tendency to cause mutagenesis at the target sites with a shorter spacer, suggesting a shorter spacer as a desirable property for increasing the specificity of the cleavage activity of TALEN. These TALENs with new structure can provide a new method for genome engineering.

### Experimental Example 10

### Development of Golden-Gate Assembly System

In the present invention, one-step Golden-Gate cloning system was developed to assemble TALEN plasmids with various lengths in a high throughput manner. Although Golden-Gate cloning methods have been previously used for assembling TALEN plasmids, those methods rely on PCR or require isolation of DNA segment from agarose gels or multiple sub-cloning steps. On the other hand, the present Golden-Gate system employs a total of 424 TALE array plasmids (6×64 tripartite arrays, 2×16 bipartite arrays, and 2×4 monopartite arrays) and 8 obligatory heterodimeric FokI-encoding plasmids. In order to make the modular array, a combination of four TALE repeat domains, namely NI, NN, NG, and HD, was used each targeting one of the four bases (A, G, T, and C, respectively). These TALE repeat domains consist of 34 amino acid residues with a high sequence homology; the amino acids at the positions 12 and 13 of RVD determine the specificity of TALEN.

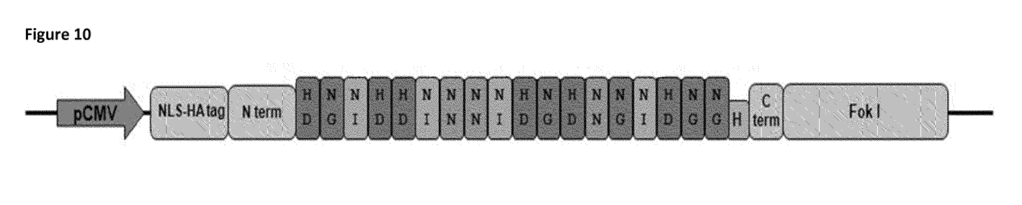

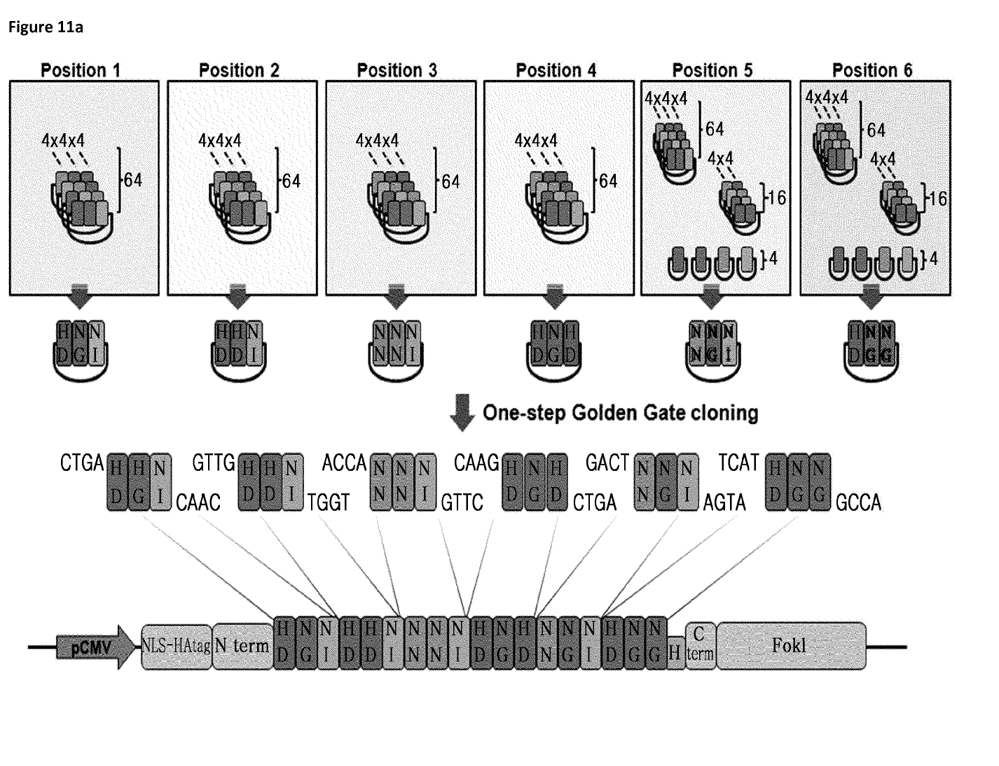

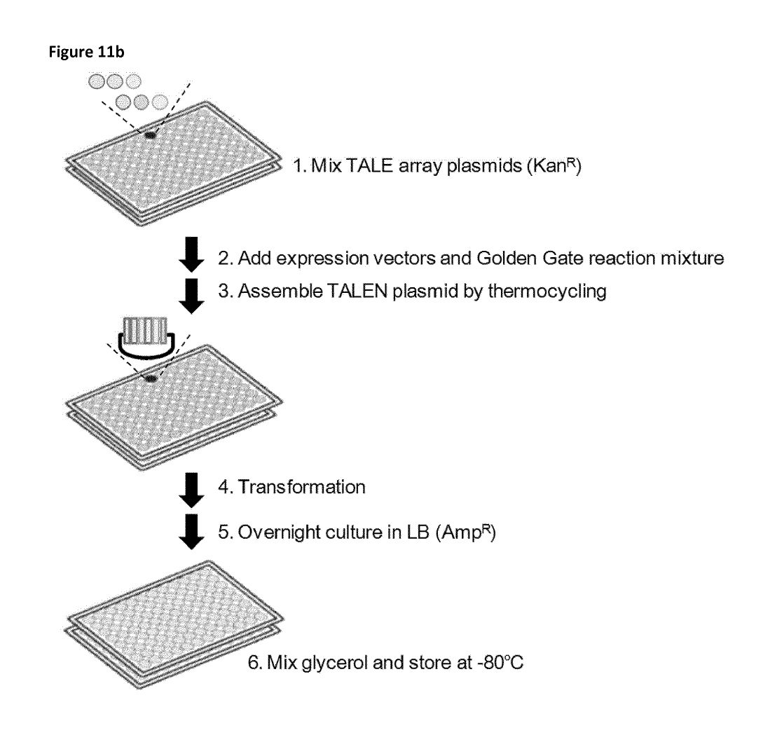

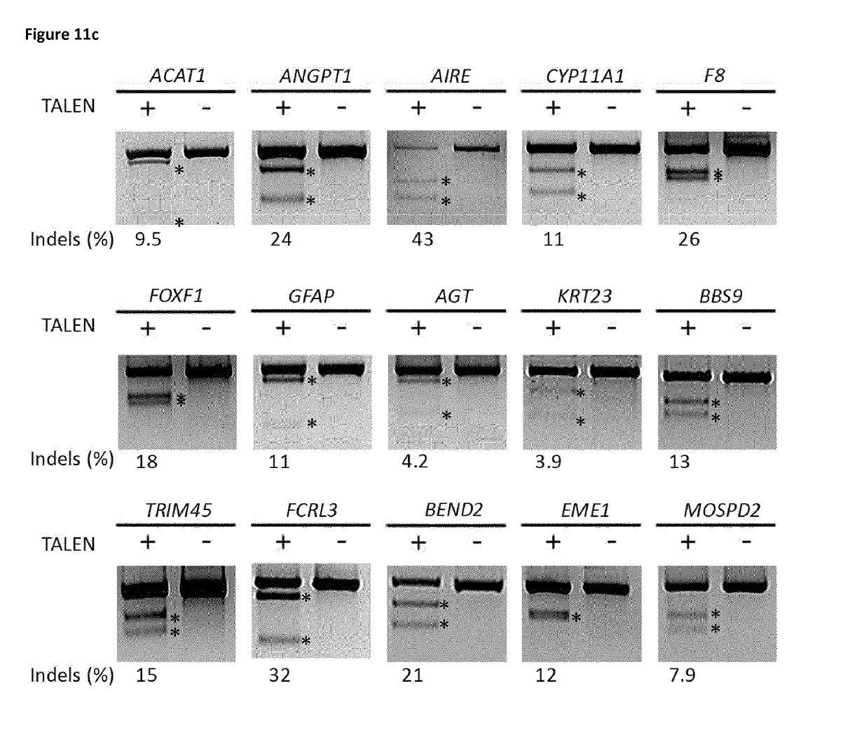

The TALE array plasmids are divided into 6 subgroups according by their positions (FIG. 10). Digestion of a TALE array with BsaI at a designated position generates the same four-base overhang but digestion at a different position generates a different four-base overhang. One RVD is chosen for each of the 6 positions; the 6 chosen RVDs are combined to be sub-cloned into one of the FokI expression plasmids (FIG. 11b). This system allows construction of TALEN plasmids that contain at least 14.5 RVD modules (=4 tripartite arrays+2 monopartite arrays) up to 18.5 RVD modules (=6 tripartite arrays) in a single Golden-Gate reaction. The gene encoding the last half-repeat is previously inserted into the FokI plasmids. These TALENs recognize DNA sequences of 16 to 20 bps in length including a conserved base T at the 5′ end. As TALENs works as a dimer, these TALEN pairs recognize 32- to 40-bp long DNA sequence that consist of two half-sites separated by a spacer with a length of 12- to 14-bp.

### Experimental Example 11

### A pilot-Scale Construction of TALENs

To determine whether the new TALEN architectures assembled by the one-step Golden-Gate system can be efficiently used for genome-editing of the cultured human cells, 15 TALEN pairs were constructed, each targeting a different human gene. Each of the TALENs consists of 18.5 RVD modules and an obligatory heterodimeric FokI domain. The genome-editing activity of these TALENs in HEK 293 cells was analyzed by using T7 endonuclease I (T7E1) which is an enzyme that specifically recognizes and cleaves heteroduplexes formed by hybridization of wild-type and mutant DNA sequences. Plasmids that encode each TALEN pair were transfected into HEK 293 cells and the genomic DNA was amplified by PCR, which was then subjected to a T7E1 assay. Mutation frequencies were determined by measuring the intensities of cleaved bands relative to intact bands. Mutations were detected at all of the 15 target sites at frequency ranging from 3.9% to 43% (FIG. 11c). This pilot experiment demonstrates that both of a new TALEN architecture and the Golden-Gate assembly system are robust enough to allow genome-scale construction of TALENs.

### Experimental Example 12

### Genome-Scale Assembly of TALENs

One target site per gene was chosen and TALEN expression plasmids were assembled using the Golden-Gate cloning system. To facilitate the process of large-scale assembly, 18.5/18.5 RVD TALEN sites with 12-bp spacers were chosen in each gene preferentially. A total of 37,480 plasmids encoding 18,740 TALEN pairs were assembled in 96-well plates according to the optimized protocol (FIG. 11b).

Quality control of the TALEN plasmids was performed by 1) digesting of plasmid with EcoRI restriction enzyme and 2) DNA sequencing. One E. coli transformant was chosen from each of the 399 96-well plates. TALEN plasmids were purified from 4 colonies that were grown from the same transformant, and then digested with EcoRI. The correct assembly of TALEN plasmid showed a 2.5-kbp band on the gel. Typically, at least 2 out of 4 plasmids isolated from each transformant showed a 2.5-kbp band demonstrating that the plasmids were assembled correctly. In order to confirm the TALE array sequence in these plasmids, a dideoxy DNA sequencing was performed for the 298 plasmids that showed an expected size of band after being digested with EcoRI, and it was found that all of these plasmids contained the expected sequences. Overall, these results confirm the robustness of the present Golden-Gate cloning system.

Then, 104 TALEN pairs targeting different genes were selected for further investigating their genome-editing activity in HEK 293 cells through T7E1 assay. Mutations were detected in 101 out of 103 target sites that were PCR-amplified (assay sensitivity of about 0.5%). Thus, the success rate of producing a correct form of TALENs was 98.1%. These TALENs were highly active: 76% (=78/103) of TALENs demonstrated a mutation frequency of greater than 5% (or indel %) while 55% (=57/103) of TALENs showed a mutation frequency of greater than 10% (FIG. 12).

The above results demonstrate that TALENs can replace ZFNs to induce site-specific genome modifications in cultured human cells. The minimal DNA-binding domain of TALEs, the linker between the TALE moiety and the FokI domain, and the spacer length at the target site were systematically defined. Both TALEN/ZFN hybrids and TALEN pairs showed genome editing activities at predetermined endogenous sites in a chromosomal context. It is expected that TALENs can be used broadly for precise genomic modifications in plants, animals, and cultured cells including human stem cells, and may add a new dimension to genome engineering by targeting sites not amenable for modifications using ZFNs.

Also, a new TALEN architecture has an enhanced target specificity and cleavage activity compared to the previous TALEN.

What is claimed is:

1. A fusion protein having nuclease activity, comprising a TAL (transcription activator-like) effector (TALE) domain and a nucleotide cleavage domain,
wherein the TALE domain includes one or more TALE-repeat modules, each of the TALE-repeat modules recognizing a single specific nucleic acid.

2. The fusion protein according to claim 1, consisting of a N-terminal domain, one or more TALE-repeat modules followed by a half-repeat module, a linker and a nucleotide cleavage domain.

3. The fusion protein according to claim 2, wherein the N-terminal domain is amino acid sequences of SEQ ID NO:28.

4. The fusion protein according to claim 2, wherein the linker is an amino acid sequence of SEQ ID NO: 60, 61 or 62.

5. The fusion protein according to claim 1, wherein the TALE domain comprise one to thirty TALE-repeat modules.

6. The fusion protein according to claim 1, wherein the TALE domain comprises 135 amino acids sequences of SEQ ID NO: 28 upstream of TALE-repeat modules.

7. The fusion protein according to claim 1, wherein the TALE-repeat module is amino acids sequence of SEQ ID NOs: 24, 25, 26, 27, 40, 41, 42, 43, 44, 45, 46, 47, 48, 49, 50, 51, 52, 53, 54, 55, 56, 57, 58, or 59.

8. The fusion protein according to claim 7, wherein the 12th and 13th amino acids of TALE-repeat module together recognize a single specific nucleic acid.

9. The fusion protein according to claim 1, wherein the TAL effector (TALE) domain and nucleotide cleavage domain are linked by a linker.

10. The fusion protein according to claim 9, wherein length of the linker is 0 to 16 amino acids.

11. The fusion protein according to claim 1, having amino acids of SEQ ID NOs: 3, 6, 9, 36, or 38.

12. The fusion protein according to claim 1, wherein the TAL effector nuclease functions as a dimer to cleave a nucleotide sequence.

13. The fusion protein according to claim 12, wherein the dimer is a homodimer of TAL effector nuclease or a heterodimer of TAL effector nuclease and zinc finger nuclease.

14. The fusion protein according to claim 1, being designed such that the length of spacer between a first half site and a second half site, which two TALE domains of the fusion protein dimer respectively bind, is 9- to 14-bp.

15. The fusion protein according to claim 2, being designed such that the length of spacer between a first half site and a second half site, which two TALE domains of the fusion protein dimer respectively bind, is 10- to 14-bp.

16. The fusion protein according to claim 1, wherein the nucleotide cleavage domain is the cleavage domain from the type IIs restriction endonuclease.

17. The fusion protein according to claim 16, wherein the type IIs restriction endonuclease is FokI.

18. A nucleotide sequence, encoding the fusion protein of claim 1.

19. A kit for cleavage, replacement or modification of nucleotide sequences in targeted region, comprising one or more pairs of the fusion proteins of claim 1.

20. A kit for cleavage, replacement or modification of nucleotide sequences in targeted region, comprising one or more pairs of the fusion proteins of claim 2.

21. A cell, comprising the fusion protein of claim 1.

22. A cell, comprising the fusion protein of claim 2.

23. A method for deletion, duplication, inversion, replacement, insertion or rearrangement of genomic DNA, comprising the step of cleaving specific sites in a genome using one or more pair of the fusion proteins of claim 1.

24. A method for deletion, duplication, inversion, replacement, insertion or rearrangement of genomic DNA, comprising the step of cleaving specific sites in a genome using one or more pair of the fusion proteins of claim 2.
